(19) United States
(12) Patent Application Publication
(10) Pub. No.: US 2008/0115238 A1
(43) Pub. Date: May 15, 2008

(54) HEDGEHOG INHIBITOR ASSAY

(76) Inventors: Rune Toftgard, Skarholmen (SE); Matthias Lauth, Huddinge (SE); Stephan Teglund, Ronninge (SE)

Correspondence Address: FISH & RICHARDSON P.C., PO BOX 1022, MINNEAPOLIS, MN, 55440-1022, US

(21) Appl. No.: 11/828,406

(22) Filed: Jul. 26, 2007

Related U.S. Application Data
(63) Continuation-in-part of application No. PCT/SE2006/000141, filed on Jan. 31, 2006.
(60) Provisional application No. 60/648,557, filed on Jan. 31, 2005.

(51) Int. Cl. A01K 67/027; C12Q 1/68; G01N 33/567; C12Q 1/66; G01N 33/53

(52) U.S. Cl. 800/18; 435/7.2; 435/6; 435/8; 435/455

(57) ABSTRACT

The present invention relates to the field of Hedgehog signalling, and more specifically to a cell-based assay system and methods for identifying inhibitors and/or antagonists of specific cellular events in said signalling pathway. The present invention hereby proposes a novel approach for identifying inhibitors and/or antagonists downstream of the signalling components Smoothened and Patched, e.g. at the Gli-level. The assay comprises cells lacking a functional Sufu protein, which according to the invention is a protein component of emerging importance in the Hedgehog signalling pathway.

### CROSS-REFERENCE TO RELATED APPLICATIONS

This application is a Continuation-in-Part application under 35 U.S.C. §111 (a) and claims benefit under 35 U.S.C. §119(a) of International Application No. PCT/SE2006/000141 having an International Filing Date of Jan. 31, 2006, which claims the benefit of U.S. Provisional Application Ser. No. 60/648,557, filed Jan. 31, 2005.

### FIELD OF THE INVENTION

The present invention relates to the field of Hedgehog signalling pathways, and to a novel approach for identifying an inhibitor and/or an antagonist of a Hedgehog signalling pathway. The present invention discloses a significant, central and previously unknown, role of the component Sufu (Suppressor of Fused) in the Hedgehog signalling pathway.

### BACKGROUND OF THE INVENTION

The Hedgehog signalling pathway was first discovered in Drosophila, is evolutionary conserved, and plays important roles in embryogenesis and carcinogenesis (2-8). In the study of the development of cells, fruit flies have extensively been used as a model, as these organisms are less complex than mammalian ones. The Hedgehog (HH) signalling pathway is evolutionary conserved from invertebrates, like Drosophila, to vertebrates, like mouse and human. In Drosophila, as well as in mice, genetic inactivation of the Hedgehog pathway leads to severe developmental malformations and embryonic lethality. On the contrary, constitutive activation results in hyperproliferation and tumours.

Pattern formation takes place through a series of logical steps, reiterated many times during the development of an organism. Viewed from a broader evolutionary perspective, across species, the same sort of reiterative pattern formations are seen. The central dogma of pattern formation has been described by Lawrence and Struhl, 1996 (1).

There are three known mammalian Hedgehog proteins, which bind to the Hedgehog receptor. These are named Sonic (SHH), Indian (IHH) and Desert (DHH) Hedgehog. The proteins can substitute for each other, but in wildtype animals, their distinct distributions result in unique activities. SHH controls the polarity of limb growth, directs the development of neurons in the ventral neural tube and patterns somites. IHH controls endochondral bone development and DHH is necessary for spermiogenesis. Vertebrate Hedgehog genes are expressed in many other tissues, including the peripheral nervous system, brain, lung, liver, kidney, tooth primordia, genitalia and hindgut and foregut endoderm.

About sixteen genes in the Hedgehog class are known. The encoded proteins include kinases, transcription factors, a cell junction protein, two secreted proteins called wingless (WG) and the above mentioned Hedgehog (HH), a single transmembrane protein called patched (Ptch), and some novel proteins not related to any known protein. All of these proteins are believed to work together in signalling pathways that inform cells about their neighbours in order to set cell fates and polarities.

When one of the three known mammalian proteins Sonic Hedgehog (SHH), Indian Hedgehog (IHH), Desert Hedgehog (DHH), with SHH being the most prominent, binds to their common receptor, Patched (Ptch1), the repressive effect of Ptch1 on another transmembrane protein, the proto-oncogene Smoothened (Smo) is relieved. The derepression of Smo results, in a yet unknown fashion, in the activation of the downstream complex of Suppressor of Fused (Sufu) and the transcription factor Gli (three Gli proteins are known in mammals: Gli-1, Gli-2, Gli-3). Gli is a component of the Hedgehog signalling pathway, which functions as a transcription factor and activates transcription of specific genes.

The Sufu-Gli complex, mentioned in the above, translocates from the cytoplasm into the nucleus and activates transcription of specific target genes. Sufu is hereby a negative interactor, as Gli alone is sufficient to activate gene transcription (9, 10).

In mammals, functions corresponding to that of the component Ci in Drosophila have been assigned to the zincfinger-containing and DNA-binding proteins Gli 1-3, mentioned in the above, which are expressed in an overlapping pattern adjacent to cells secreting Sonic Hedgehog, or the homologous Indian Hedgehog and Desert Hedgehog.

Mutations in the gene encoding Gli-3 detected in human disorders, result in expression of truncated Gli-3 proteins that mimic natural Ci processing in Drosophila with respect to their altered subcellular localization and transactivation properties in HeLa cells (the full-length protein is cytoplasmic) (11). In the case of Gli-1, initial studies using the D259MG glioma cell line, which contains an amplified Gli-1 locus, showed that this protein is nuclear in localization (12); nuclear Gli-1 localization was also seen after Gli-1 cDNA was transfected into COS cells (13). In contrast, Gli-1 in human basal cell carcinomas (BCCs) was again cytoplasmic (13).

More recently it has been shown that, upon overexpression, all three Gli proteins may show either cytoplasmic or nuclear localization, depending on the cellular context (14). Analysis of the proteolytic processing of vertebrate Gli proteins using mouse embryo extracts has shown the appearance of shorter variants of endogenous Gli-3, but not of Gli-1 (15), whereas after overexpression in frog embryos, shorter variants of all three Gli proteins were observed (14). However, as overexpressed full-length Gli proteins can be detected in the nucleus, proteolytic processing does not appear to be necessary for nuclear import. Taken together, these data strongly indicate that, in mammalian cells, there may be mechanisms that regulate the subcellular localization of both full-length and processed Gli proteins. Such mechanisms could involve the regulation of interactions of the Gli proteins with anchoring proteins, or the modification of the Gli proteins themselves, including proteolytic processing.

Hence, the Hedgehog signalling pathway is of key importance for both normal development and carcinogenesis, as shown by the presence of mutations in genes encoding components of this pathway in human malformation and cancer-prediposing syndromes (holoprosencephaly (14), nevoid basal cell carcinoma syndrome (16-18).

Additionally, several types of cancers have been linked to aberrant activation of the Hedgehog pathway, such as Basal Cell Carcinoma (BCC), Trichoepitheliomas, Medulloblastoma, small-cell lung cancer, bladder carcinoma, and very recently, digestive tract, pancreas and prostate malignancies (4, 6-8, 19, 20). There are also reports about increased Hedgehog signalling in keloids, and interestingly, one study suggesting that Hedgehog antagonists can cause disappearance of psoriatic plaques.

Hedgehog signalling may also be implicated in rhabdomyosarcoma (21), esophaegal carcinoma (22), stomach adenocarcinoma (23), liver cancer (24), pericytoma (25), breast carcinoma (26), glioma (27), plasmacytoma (28), ovarian fibroma (29) as well as in stem cell proliferation (30), hair growth (31) and body weight/size (32).

The apparent involvement and importance of the Hedgehog signalling pathway in the onset of several cancer forms has of course generated a lot of interest in identifying inhibitors of this pathway.

The present inventors are able to show the emerging importance of the Suppressor of fused (Sufu), an intracellular component of the Hedgehog signalling pathway. Sufu has a proposed role in nuclear shuttling of the Ci/Gli transcription factors (9, 33-35) and is shown to suppress the effects of mutations in the kinase Fused, but having essentially no detectable phenotype, when eliminated alone in the fly (36, 37).

Patent application nr. EP1482929 discloses available methods and reagents for inhibiting aberrant growth states resulting from Hedgehog gain-of-function, Ptch loss-of-function or Smoothened gain-of-function comprising contacting the cell with a Hedgehog antagonist, such as a small molecule, in a sufficient amount to aberrant growth state, e.g. to agonize a normal Ptch pathway or antagonize Smoothened or Hedgehog activity. However, the patent application does not disclose methods for selectively inhibiting events downstream of Smo and/or Ptch, or using cell deprived of a functional Sufu protein.

Also WO0236818 discloses substances that block Hedgehog signalling through modifications of Ptch and Smo vesicular sorting for the preparation of medicaments for the treatment of a mammalian cancer. Furthermore, U.S. Pat. No. 6,261,686 discloses an assay for identifying substances able to potentiate or inhibit binding of a Hedgehog polypeptide to a naturally occurring Ptch receptor.

The publication “Identification of a small molecule inhibitor of the Hedgehog signalling pathway: effects on basal cell carcinoma-like lesions” (38) concerns an assay wherein Ptch has been deleted, targeting the upstream events of the Hedgehog signalling pathway with a specific inhibitor.

Consequently, the focus of the field has however until now been on targeting the Smo and Ptch components of the Hedgehog signalling pathway with inhibitors. However, examples exist in literature of cell tumors/cell lines having increased Hedgehog signaling but lacking mutations in Ptch1 or Smo and not showing increased ligand expression (7), consequently rendering available drugs non-functional in such cells. Hence, in light of prior art, there is a need to develop an assay which makes it possible to identify substances which are able to target signaling events further downstream in the Hedgehog signaling pathway, as the inventors have shown that not only Smo and Ptch, but the component Sufu plays an important role in the pathway.

### SUMMARY OF THE INVENTION

The present invention relates to a novel cell-based assay system for identifying inhibitors and/or antagonists of a Hedgehog signalling pathway downstream of the Hedgehog signalling components Smoothened (Smo) and Patched (Ptch). The present invention also brings forward a novel method for identifying inhibitors and/or antagonists of a Hedgehog signalling pathway, which will selectively target components downstream of the Hedgehog signalling component Sufu, and/or components dependent upon Sufu. This approach offers a new way of identifying inhibitors and/or antagonists, which are not dependent upon the Smo/Ptch pathway, but which pathway continues downstream of Sufu, or via an alternative way to Sufu, to the Gli level. The present invention provides the possibility and advantage of identifying substances which would selectively target cells with a non functional Sufu protein, which have been shown to be present in certain tumors, as mentioned in the above. Thus, such a method can comprise adding a substance to be tested to a cell-culture comprising cells lacking a functional Sufu protein, and detecting whether or not the substance has an inhibiting and/or antagonizing function on a Hedgehog signalling pathway in the cells. The cells lacking a functional Sufu protein can be generated by deleting the Sufu gene from the genome in the cells. The method can comprise adding a substance to be tested to Sufu−/− cells transfected with a reporter gene system responsive to a Hedgehog signalling gene product, lysing the cells, and measuring the levels of reporter gene expression and/or activity to detect whether or not the substance has an inhibiting and/or antagonizing function on a Hedgehog signalling pathway in the cells. The reporter gene system can comprise a plasmid for normalization. The reporter gene system can be responsive to Gli-mediated transcription of a gene. The reporter gene system can comprise a reporter gene comprising Gli binding sites, e.g., the reporter gene system can comprise 8 Gli binding sites and a phRL-SV40 or a phRL-TK plasmid. The method can comprise adding a substance to be tested to untransfected confluent Sufu−/− cells, preparing DNAse-treated RNA from the cells and reversibly transcribing the RNA into cDNA, performing real-time PCR with the cDNA for the detection of Hedgehog target gene mRNA, and normalizing the level of a Hedgehog target gene mRNA against the level of a housekeeping gene mRNA using a relative and/or quantitative method to detect whether or not the substance has an inhibiting and/or antagonizing function on a Hedgehog signalling pathway in the cells. The Hedgehog target gene mRNA can be Gli1 mRNA. The normalization can be performed against Gapdh mRNA levels using a standard curve of a Gli1 positive sample to detect whether or not the substance has an inhibiting and/or antagonizing function on a Hedgehog signalling pathway. The method can include detecting whether or not the substance induces cell death by necrosis or apoptosis in the cells.

Hence, the present invention is based on selectively using cells, which lack a functional Sufu protein, thereby targeting inhibitors of downstream components of a Hedgehog signalling pathway. A Sufu protein and/or a Sufu gene is deleted, or made not functional, in the cells in any suitable way. In one aspect of the invention, a cell-based assay is used, which comprises Sufu^{-/-} cells. In a particular embodiment, such an assay comprises mouse embryonic fibroblasts. Of course any other cells suitable for the purpose, and which lack a functional Sufu protein, may also be used in the present context. Such cells include, without limitation, prokaryote cells and eukaryote cells such as yeast cells, insect cells or mammalian cells.

The cells lacking a functional Sufu protein can be used in a method using a reporter gene system, or equally preferred, in a real-time PCR based system, to detect the effect on Hedgehog signalling of the inhibitor and/or antagonist tested. Other suitable methods may also be used, wherein a Sufu protein and/or Sufu gene has been deleted from the cells.

Additionally, a mouse model is disclosed, which possess a Sufu^{-/-} genotype. This mouse may also be of use for identifying inhibitors and/or antagonists of a downstream event of a Hedgehog signalling pathway, downstream of Smo and Ptch, as well as for investigating an effect of an inhibitor and/or an antagonist. Said animal model may also be used for investigating an effect of such an inhibitor and/or antagonist in a given disease model. A method for preparing an immortalized mouse embryo fibroblast Sufu−/− cell culture comprises intercrossing chimeric Sufu+/− mice comprising a vector that targets a Sufu locus to generate Sufu−/− mice embryos, selecting Sufu−/− embryos, and incubating cells obtained from the Sufu−/− mice embryos in MEF medium, thereby preparing the immortalized mouse embryo fibroblast Sufu−/− cell culture. The vector can be pSufuΔexon1neo. Also disclosed is a transgenic mouse lacking a functional Sufu protein.

The present approach was developed based on the present inventors surprising findings of the emerging importance and central role of the component Sufu in the Hedgehog signalling pathway. Earlier attempts of identifying inhibitors of the Hedgehog signalling pathway have been focused on targeting the Smoothened (Smo) and Patched (Ptch) components, further upstream in the signalling pathway.

### BRIEF DESCRIPTION OF THE DRAWINGS

FIG. 1: Simplified picture of a Hedgehog signal transduction. In the absence of ligand (left), Ptch inhibits Smo, which in turn does not signal to its downstream effector Gli. In unstimulated cells, Gli is inactive and localized mainly to the cytoplasm and thus target gene transcription is off. In the presence of ligand (Sonic Hedgehog (SHH), Desert Hedgehog (DHH) or Indian Hedgehog (IHH), summarized as HH in the figure), Ptch inhibition of Smo is alleviated and Smo signals to Gli. Gli becomes activated, translocates into the nucleus and induces target gene transcription. Other components of the pathway are omitted for clarity.

FIG. 2: Sufu gene targeting strategy creates a null allele. (A) Homologous recombination in ES cells with targeting vector pSufuΔexon1^{neo} replaces exon 1 of the Sufu gene with a neomycin resistance cassette. Immediately upstream of the Sufu gene is the gene α-centractin 1a (Actr1a) located in a head-to-head orientation. The locations of primers used are indicated with small arrows (see Table 1 for primer sequences). S, SacI; E, EcoRI; H, HindIII; Xb, XbaI; B, BamHI; V, EcoRV. (B) Genotyping of E9.5 embryos from a Sufu^{+/-} intercross using Southern blot hybridization with a 400-bp EcoRI/EcoRV 3′ external probe that hybridizes with a 6.3-kb and a 17.5-kb wild-type and mutant SacI fragment, respectively. (C) Genotyping of E9.5 embryos from a Sufu^{+/-} intercross using PCR with the F21/R6/Neo F2 primers that amplifies a 274-bp and 194-bp wild-type and mutant Sufu allele, respectively. (D) Northern blot analysis of total RNA from E8.5 and E9.5 embryos showing absence of Sufu mRNA in Sufu^{-/-} embryos (lanes 3 and 6) and a 50% reduction in Sufu^{+/-} embryos (lanes 2 and 5). As loading control, re-probing the membrane with a β-actin probe was used. (E) Western blot analysis of total cell lysate from E9.5 embryos showing absence of Sufu protein in Sufu^{-/-} embryos (lane 3) and a 50% reduction in Sufu^{+/-} embryos (lane 2). As loading control, re-probing the membrane with antisera against β-actin was used. (F) RT-PCR analysis (25 cycles) of the neighbouring gene Actr1a in Sufu wild-type (+/+) and Sufu mutant (−/−) E9.5 embryos shows no gross disturbance of its expression.

FIG. 3. Sufu^{-/-} embryos die in utero at around E9.5 with failure of completely closing the neural tube and cephalic vesicles. (A, D and G) Wild-type (Wt), Sufu^{-/-}, and Ptch1^{-/-} E9.5 embryos. (B, E and H) SEM analysis showing the open hindbrain and the anterior neuropore (arrows) in the Sufu^{-/-} and Ptch1^{-/-} embryos. (C, F and I) Formalin-fixed and hematoxylin-eosin stained transverse sections from the cephalic region showing lack of closing and properly forming the cephalic vesicles in both Sufu^{-/-} and Ptch1^{-/-} embryos. However, the anterior Sufu^{-/-} neuroepithelium forms a wider, more open structure with considerably less mesenchymal tissue compared to the Ptch1^{-/-} embryos. Anterior is to the left and posterior to the right. Scale bar=100 μm.

FIG. 4. Ectopic activation of Hh target genes and aberrant expression of other Hh pathway components in Sufu^{-/-} embryos mimicking that seen in Ptch1^{-/-} embryos. (A-R) Whole-mount in situ hybridizations of wild-type (Wt), Sufu^{-/-}, and Ptch1^{-/-} E9.5 embryos with DIG-labeled anti-sense riboprobes against Shh (A-C), Ptch1 (D-F), Sufu (G-I), Gli1 (J-L), Gli2 (M-O), and Gli3 (P-R).

FIG. 5. Ventralization of the Sufu^{-/-} embryonic neural tube similar to that seen in Ptch1^{-/-} embryos. (A-L) Transverse sections of the neural tube at the thoracic level of wild-type (Wt), Sufu^{-/-}, and Ptch1^{-/-} E9.5 embryos immunofluorescently stained with antibodies against Shh (red) (A-C), FoxA2 (red) and Isl1/2 (green) (D-F), Nkx2.2 (green) and Pax6 (red) (G-I), and Nkx6.1 (green) (J-L). (M-R) In situ hybridization of neural tube sections of Wt and Sufu^{-/-} E9.5 embryos hybridized with DIG-labeled riboprobes against Ptch1 (M and N), Gli3 (O and P), and Nato3 (Q and R).

FIG. 6. GLI1 predominantly reside in the cytoplasm of Sufu^{-/-} MEFs, whose constitutive Hh pathway activity cannot be modulated at the level of Smo and can be partially blocked by PKA. (A) Agarose gel analysis of RT-PCR reactions from Sufu^{+/+} (Wt), Sufu^{-/-} cell line #1 and Ptch1^{-/-} MEFs with primers specific for Ptch1, Ptch2, Smo, Sufu, Gli1, and Gli2. Primers for Hprt were used as control for RNA input levels. (B) Transient transfection of Sufu Wt (+/+) and mutant (−/−) MEF lines #1 and #2 with 8xGliLuc (filled columns) or 8xGli^{mut}Luc (open columns) luciferase reporter plasmids with (+) or without (−) the pMYC-SUFU expression plasmid. Gli reporter activity is presented relative to Wt MEFs, which was given an arbitrary level of 1.0, after compensating for transfection efficiency using the renilla luciferase reporter plasmid. Results are means ±standard deviation (SD) of the mean of at least three independent experiments. (C) Treatment of Wt, Sufu^{-/-} cell line #1 and Ptch1^{-/-} MEFs with 10 μM cyclopamine (shaded column), 100 nM Smoothened agonist (SAG) (black column) or with DMSO alone (open column) for two days prior to measuring 8xGliLuc reporter activity. (D) Treatment of Wt, Sufu^{-/-} cell line #1 and Ptch1^{-/-} MEFs with 1 μM of the PKA-inhibitor H-89 (light shaded columns), 100 μM forskolin (dark shaded column), transfected with a constitutively active PKA subunit expression plasmid (black column) or untreated (open column) for one day prior to measuring 8xGliLuc reporter activity. (E) Confocal images of Wt, Sufu^{-/-} cell line #1 and Ptch1^{-/-} MEFs after transient transfection with expression plasmids for EGFP::GLI1 or EGFP alone for 24 hours with or without 10 ng/ml Leptomycin B (LMB) treatment for 6 hours. Nuclei are stained blue.

FIG. 7. Sufu^{+/-} mice develop a skin phenotype with Gorlin-like features. (A and B) Ventral view of a wild-type (Wt) and a Sufu^{+/-} mouse, the latter showing alopecia and increased pigmentation. (C and D) Paws from a Wt and a Sufu^{+/-} mouse, the latter displaying increased pigmentation and skin papules. (E and F) Tails from a Wt and a Sufu^{+/-} mouse, the latter with increased pigmentation and skin nodules. (G and H) Hematoxylin-eosin (H&E) stained paw sections from a Wt and a Sufu^{+/-} mouse, the latter showing several epidermal basaloid proliferations (arrow-heads). Scale bar=100 μm. (I and J) Paw tissue sections from a Wt and a Sufu^{+/-} mouse immunostained against Ki67, the latter demonstrating relatively few positive cells in the epidermal proliferations (arrows). Scale bar=100 μm. (K and L) H&E-stained jaw sections from a Wt and a Sufu^{+/-} mouse, the latter with a keratocyst (arrow). Scale bar=50 μm. All mice are around two years of age.

FIG. 8. Sufu^{+/-} skin develops basaloid follicular hamartomas and aberrant sebaceous gland morphology. (A and B) Skin tissue sections from the paw of a wild-type (Wt) and a Sufu^{+/-} mouse immunostained for Keratin 5 (K5) showing strong and relatively uniform expression in the Sufu^{+/-} proliferations. (C and D) Skin tissue sections from the paw of a Wt and a Sufu^{+/-} mouse immunostained for Keratin 6 (K6) showing strong but heterogeneous expression in the Sufu^{+/-} proliferations. (E and F) Skin tissue sections from the paw of a Wt and a Sufu^{+/-} mouse immunostained for Keratin 17 (K17) showing strong expression in the Sufu^{+/-} proliferation, particularly in those cells outlining the proliferation. (G) Hematoxylin-eosin (H&E)-stained tissue section from the axillary region. Arrows denote branching hyperplastic sebaceous glands. (H) H&E-stained tissue section from the tail showing extensively branched proliferations. Inset highlights condensed fibroblastoid cells forming a dermal papilla-like structure resembling an abortive hair follicle formation (arrow). (I) Dorsal skin section immunostained for K6 demonstrating, as in (D), heterogeneous immunoreactivity. Inset shows a K6-negative region of the proliferation. (J) The histological aberrant sebaceous glands express Indian Hedgehog (Ihh). All images are from Wt or Sufu^{+/-} mice around two years old. Scale bars=100 μm (A-G, I and J); 200 μm (H); 50 μm (insets of H,I).

FIG. 9. Skin from Sufu^{+/-} mice express increased levels of Gli1 indicative of an active Hh pathway and genetic diagrams depicting the divergence of core components in the pathway in insects versus mammals. (A and B) Real-time quantitative PCR for Gli1 expression in Wt (filled columns) and Sufu^{+/-} (open columns) skin around one-year old mice on B6 (A) genetic background (generation N7) or two-year old mice on mixed B6;129 background (B). Gli1 expression is presented relative to Wt skin, which was given an arbitrary level 1.0, after normalizing the samples using the mouse GAPDH as endogenous control. Results are means ±standard deviations of samples in triplicate. (C) In Drosophila, the major pathway controlling Ci downstream of Smo is mediated via Cos2 while the pathway via Sufu has a less important role. In the mouse, a major post-receptor mechanism controlling Gli activity is mediated by Sufu, as we have demonstrated in this invention.

FIG. 10. Inhibition of GLI-induced transcription. (A) Sufu^{-/-} MEFs were transfected with Hh/Gli reporter plasmid 8xGliLuc and a renilla luciferase plasmid for normalisation. Luciferase activity was measured after 72 h of treatment time with the indicated compound. GANT (Gli-ANTagonist) 55 (2-(3-Dimethylamino-propylamino)-anthraquinone) reduces signal intensity in a dose-dependent manner, whereas Cyclopamine (Cyclo) is ineffective. The structure of GANT55 is given on the right. (B) Gli1 inhibition, (C) Gli2 inhibition in HEK293 cells. Treatment was for 48 h after transfection of the corresponding Gli plasmid and a Hh/Gli reporter (12xGliBS-Luc) and a renilla luciferase plasmid for normalisation. Note that in all these cases the Smo inhibitor Cyclopamine is inactive since activation of the pathway occurs downstream.

FIG. 11 Dose-response curve of GANT55 in comparison to Cyclopamine in (A) Ptch1−/− MEFs. Due to genetic replacement of the Ptch1 gene by β-Galactosidase, these cells show constitutive Hh signalling. Endogenous Ptch-lacZ was used as read-out for pathway activity. Normalization was done by cotransfection of a renilla luciferase plasmid. (B) The effects seen in (A) are not due to unspecific inhibition of the reporter enzyme as could be shown by expression of a Gli-independent β-Galactosidase construct (pSV40-lacZ).

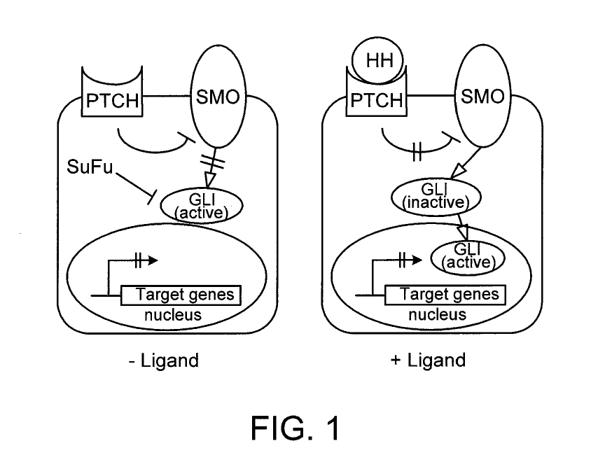

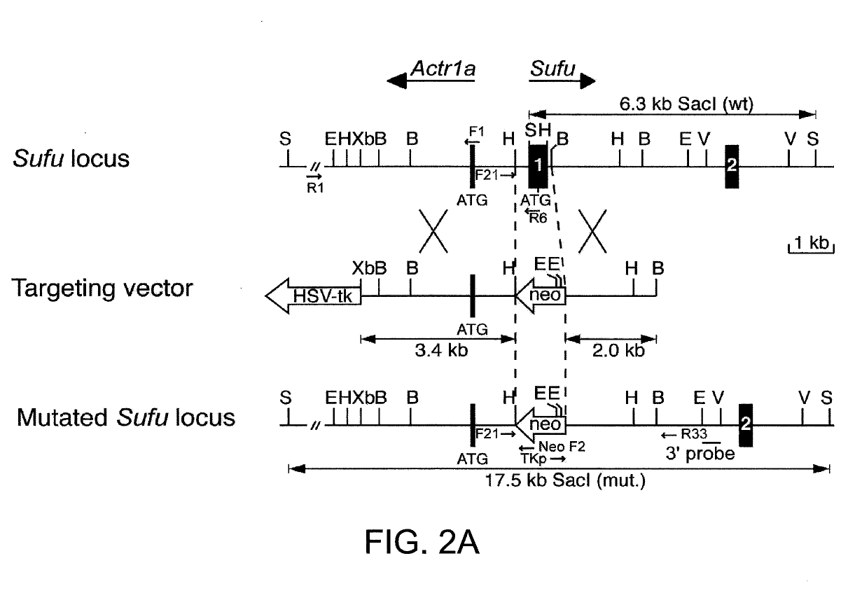

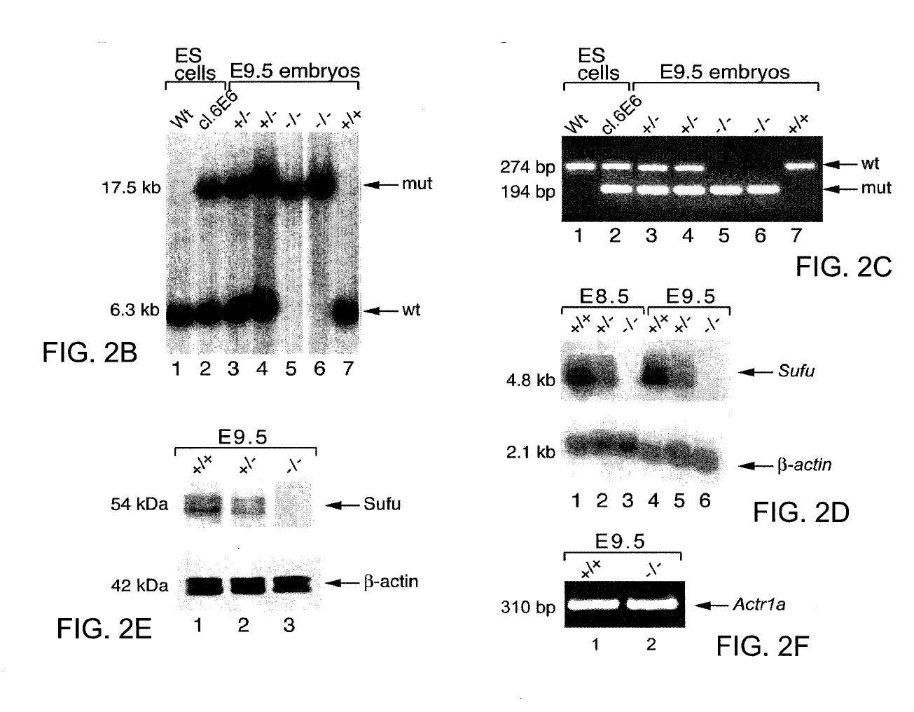

### DEFINITIONS

A “Hedgehog signalling pathway” is a signalling pathway involved in embryogenesis and carcinogenesis in many organisms, such as mammals, which is well known in the art. Said inhibitor and/or antagonist identified by a method according to the present invention is useful for inhibiting any components of such a Hedgehog signalling pathway, such as Gli1-3, Fused etc.

A “cell-based assay system”, refers to an assay system, such as a screening assay system, which comprises cells, such as mammalian or murine cells, or any other cells, excluding human embryonic stem cells, which are used in a method according to the invention, for analyzing the effect of a substance added to said cells. Said cell-based assay system is used for detecting the effect of a broad variety of substances on a Hedgehog signalling pathway in said system, which substances may be antagonists and/or inhibitors of a Hedgehog signalling pathway. An effect on a Hedgehog signalling pathway caused by a substance tested according to the invention refers to any disturbing, inhibiting, interfering, antagonizing and/or inactivating effect of the pathway. In the present context, the term “cell-based system” also comprises cells which are used to detect the effect of a substance on a Hedgehog signalling pathway in a cell lacking a functional Sufu protein, such as Sufu^{-/-} cells.

A “screening method”, an “assay” and/or a “method” may be used interchangeably in the context of the present invention.

In the context of the present invention, the terms “inhibitor”, “substance and “antagonist” may be used interchangeably, and refers to compounds which interact with the Hedgehog signalling pathway These may be selected from a broad variety of organic substances, either of natural origin such as proteins, enzymes, nucleotides, hormones, vitamins, polysaccharides, or synthetic chemical compounds such as peptides, peptidomimetics, mono or oligosaccharides or heterocycles containing approximately 1-5 aromatic or nonaromatic rings, etc, but is not limited thereto.

Cells, which in accordance with the present invention “lack a functional Sufu protein”, are cells, which by any means have lost a functional Sufu protein. This may e.g. be achieved by targeting Sufu on either a gene or a protein level, e.g. by RNAi technology, anti-sense technology, ribozymes, antibody injection, dominant negative variants of Sufu, blocking Sufu activating enzymes, and/or by using targeting antibodies, and/or by the preparation of immortalized cells in accordance with the present invention. Any suitable means may be used to delete a Sufu protein and or a Sufu gene from said cells. Such cells may be used in any embodiment according to the present invention. In the present context, cells lacking a functional Sufu protein which are used in any embodiment of the present invention, are cells in which a Sufu protein and/or a Sufu gene has been functionally deleted, and/or reduced, or cells in which the levels of Sufu protein and/or mRNA have been significantly reduced and/or abolished. “Sufu^{-/-} cells” is referring to cells which lack a functional Sufu protein. Such cells may e.g. be produced in an intercrossing between Sufu^{+/-} mice, generating an embryo with a Sufu^{-/-} genotype, i.e. both alleles of the gene have been deleted from the genome. Said Sufu^{-/-} cells generated by such an intercrossing, are unable to express a Sufu protein. “Mouse Sufu^{-/-} embryonic fibroblasts” refers to mouse embryonic fibroblasts lacking a functional Sufu protein, which may be cultivated in a medium, which medium promotes growth of said fibroblasts, such as a MEF medium. Said Sufu^{-/-} cells may be used in an assay according to the invention to test the apoptosis-inducing effect of a substance on cells deprived of a functional Sufu protein.

“DNA” refers to a deoxyribonucleic acid, which is a polynucleotide formed from covalently linked deoxyribonucleotide units, serving as the carrier of genetic information.

The term “RNA” refers to a ribonucleic acid, which is a polymer formed from covalently linked ribonucleotide monomers. Examples of RNA are mRNA and rRNA.

The term “genome” refers to the totality of genetic information belonging to a cell or an organism, and the DNA that carries this information.

The term “polypeptide”, “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. Such amino acid polymers may constitute an inhibitor and/or an antagonist identified by a method in accordance with the present invention.

The term “nucleic acid” refers to a deoxyribonucleotide or ribonucleotide (DNA or RNA) polymer in either single- or double-stranded form, and unless otherwise limited, also encompasses known analogues of natural nucleotides that can function in a similar manner as naturally occurring nucleotides.

The term “locus” refers to the position of a gene on a chromosome. Different alleles of the same gene all occupy the same locus.

“Transfected” and/or “transfection” is referring to a procedure, wherein a foreign DNA molecule is introduced into an eukaryotic cell, usually followed by expression of one or more genes of the newly introduced DNA. A transfection is performed as a part of an experiment involving a reporter gene system, in one aspect of the invention.

“Transducing” and/or “transduction” refers to introduction of a nucleotide molecule of any length, such as, but not limited to, RNA or DNA, into a cell. Such a nucleotide molecule may be introduced in the form of a free nucleotide molecule, in combination with a plasmid or carried by a viral vector, or by any other suitable means. Transduction can refer to the process by which bacterial DNA is moved from one bacterium to another using a bacterial virus (a bacteriophage, commonly called a phage).

A “reporter gene system” is a gene system which makes it possible to detect the presence of certain components e.g. in a cell. Such a system can comprise one part which recognizes the components to be detected, and one part which is triggered by such a presence through the recognition part, and which thereby produces a product which is easily detectable. Such a product is e.g. detectable by a characterising fluorescent colour, but may also be detected by other means. Examples of reporter gene systems, which may be used in the context of the present invention, are e.g. Luciferase detection systems, LacZ detection systems, Alkaline Phosphatase detection, GFP (Green Fluorescent Protein) detection and/or a CAT assay (chloramphenicol acetyltransferase). It should be noted that the present invention is however not limited to the use of the above mentioned reporter gene systems.

“Lysing” a cell, or “lysis” of a cell, is referring to the rupture of the cell's plasma membrane, leading to the release of the cell's cytoplasm, and ultimately to the death of the cell.

The term “plasmid” refers to a small circular DNA molecule that replicates independently of the genome. A plasmid is commonly used as a vector for DNA cloning. In the context of the present invention, a plasmid may be used as a part of a reporter gene system. The person skilled in the art will understand that any plasmids may be used in the present invention for transferring genetic material into a cell, such as, but not limited to, a phRL-SV40 plasmid (Promega).

An “antibody” refers to a polypeptide, substantially encoded by an immunoglobulin gene or immunoglobulin genes, and/or fragments thereof, which specifically bind and recognise an antigen. The recognised immunoglobulin genes include the kappa, lambda, gamma, delta, epsilon and mu constant regions, as well as the myriad immunoglobulin variable region genes. An exemplary immunoglobulin (antibody) structural unit comprises a tetramer. The term antibody as used herein also includes antibody fragments either produced by the modification of whole antibodies or those synthesised de novo using recombinant DNA methodologies. An antibody may be used in any suitable method according to the present invention, for example, but not limited to, targeting of a Sufu protein in a cell by antibody injection, generating a cell lacking a functional Sufu protein, and/or to detect a marker and/or a label on the surface of a cell in a FACS method.

“Gli-mediated transcription” of a gene, is in the context of the present invention referring to a gene being transcribed due to the presence of the Hedgehog signalling component Gli. In a reporter system, as disclosed by the present invention, one part of the system can recognize the presence of Gli, then another part of the system, a gene, is transcribed generating a product, which can be detected and also be quantified. Such a product can e.g. be detected by fluorescence. Gli-mediated transcription can be used in an aspect of the present invention as a part of a reporter system.

In the context of the present invention, “Gli binding sites” are referring to binding sites, which are placed in the promoter in the 5′-region of a reporter plasmid in a reporter gene system, to which Gli binds to when it is present in the cell. This binding detects the presence of Gli by triggering the transcription of a gene in said reporter plasmid, which product can be detected. Such a reporter gene system can have any suitable amount of Gli binding sites, such as between 1-5, 5-10, 10-15, or 15-20, such as 1, 2, 3, 4, 5, 6, 7, 8, 9, 10, 12, 15, 17 or 20 binding sites. In a preferred embodiment, a reporter gene system with 8 Gli binding sites is used in the context of the present invention, also named 8xGli.

Cells which have reached “confluency”, or which are “confluent”, is referring to cells, which have established a cell-to-cell contact, meaning that approximately all cells in a culture are in contact with at least one other cell in said culture. The time to reach confluency in a culture can vary, but can be between 1-5 days.

“DNAse-treated RNA” is referring to a RNA molecule, which has been treated with an enzyme named DNAse, which is characterized by its ability to fragmentize DNA, for the purpose of removing any traces of remaining DNA in a sample, which may be a reason for contamination of the sample.

“cDNA” refers to a complementary DNA molecule, which is complementary to an RNA molecule and which can be generated by reverse transcription from RNA.

The term “Reversibly transcribe” or “reverse transcription”, is in the present context referring to the process of transcribing a RNA molecule to a DNA molecule, thereby generating a complementary DNA (cDNA) molecule. Most commonly, an enzyme named reverse transcriptase performs a reverse transcription reaction.

The term “real-time PCR” is well-known in the art and is herein used to indicate a procedure, which is used to in real-time, i.e. continuously and instantly, observe fluorescence signals resulting from hybridization in conjunction with the polymerase chain reaction (PCR), and not in separate steps in said sample. This enables to instantly detect the presence of a product in a sample. Real-time PCR is described for example in U.S. Pat. No. 6,174,670. For PCR-methods in general, see for example, the techniques described in (39, 40). A real-time PCR reaction can in accordance with the present invention be performed to detect the presence, and/or quantify the expression, of a component in a Hedgehog signalling pathway in a cell. “Quantitative PCR” refers to a polymerase chain reaction (PCR) which is performed in a quantitative manner, i.e. the amount of nucleic acid molecules generated by said reaction is quantified. A quantitative PCR reaction can be a real-time PCR reaction, were quantification often is made by the detection of fluorescence as the reaction proceeds. Any quantitative PCR reaction using the appropriate conditions may of course be used in the context of the present invention.

“Hedgehog target gene mRNA”, in the context of the present invention, refers to any mRNA encoding any of the signalling components of the Hedgehog signalling pathway, such as Ptch1, Ptch2, Gli1 and/or Hip, or any other component which may be considered a target for identifying the presence of an active Hedgehog signalling pathway.

“Patched” or “Ptch” refers to a component which is part of the Hedgehog signalling pathway. Patch may refer to Ptch1 and/or Ptch2, and to a DNA (a gene), mRNA and/or a protein encoding such a component.

“Gli” refers to a signalling component in a Hedgehog signalling pathway, which may be either Gli1, Gli2, and/or Gli3, and which may refer to DNA (a gene), mRNA and/or a protein encoding such a component.

“Housekeeping genes” refers to genes, which serve a function required in nearly all cell types of an organism, regardless of their specialized role or differentiation. In a method according to the present invention, the expression of housekeeping genes is used to perform a so called “normalization” against. In such a “normalization method” a relative and/or quantitative method is used to detect the level of inhibition and/or antagonizing function caused by a substance tested by measuring the levels of expression of Hedgehog target gene mRNA, and thereafter compare the level of expression of the target gene with the level of expression of housekeeping genes. Housekeeping genes, which can be used in such a method, may be selected from the group consisting of for example Gadph, or any other housekeeping genes such as, but not limited to, PGK, β-actin, cyclophilin, and/or HPRT (Hypoxanthine-Guanine Phosphoribosyl Transferase).

In the context of the present invention, a “relative and/or quantitative method” is performed by detecting the presence and/or amount of housekeeping gene mRNA in a sample, which is subsequently compared with the presence and/or amount of mRNA from a Hedgehog target gene. Such a method may compare relative amounts of the respective mRNA, or quantify respective mRNA. Such methods may comprise methods well-known in the art, such as, but not limited to, detection by using UV-light, Northern Blot, and/or a RNase protection assay (RPA).

“Immortalized” cells are cells, which have been made immortal by various processes, such as, but not limited to, passaging through crisis, Telomerase overexpression, or growth in defined media [see e.g. (41)].

The term “chimeric mice” is used to describe mice, which are rendered by combining embryos with different genotypes, or by combining a stem cell from one embryo with one genotype with a stem cell from another embryo with another genotype, to generate a transgenic animal. The presently described technique comprising stem cells, excluding human embryonic stem cells, utilizes a tissue culture system e.g. as disclosed in an example in the experimental section of the present invention, to modify and select embryonic stem cells that carry an exogenous DNA of interest. Once derived and characterized, embryonic stem cell clones may be transferred into a few days old mouse embryos where they can differentiate into adult tissue. Preferred methods of producing pre-implantation chimeras according to the present invention are blastocyst injection of ES cells and aggregation chimeras that are produced by fusion of early stage embryos (morula) with ES cells.

A “MEF medium” (Murine Embryonic Fibroblasts) is a medium, which promotes growth of mouse embryonic fibroblasts, and can be used in a method in accordance with the present invention. Such a medium contains D-MEM (high glucose, w/o sodium pyruvate), heat-inactivated 10% fetal bovine serum (FBS), L-glutamine, sodium pyruvate, and 10 μg/ml gentamicin. As will be understood by the skilled artisan, the amounts of such ingredients may vary depending on the conditions used in such an experiment.

A “targeting vector” in the context of the present invention, refers to a vector, which targets a certain locus in a genome by inserting itself into said locus. In the context of the present invention, such a target locus is a Sufu locus. An example of such a targeting vector is pSufuΔexon1^{neo}, as disclosed in the experimental section, but any targeting vector may be used for the purpose of targeting a Sufu locus.

“RNAi technology” refers to a technology of RNA interference which may be described as a process of sequence-specific post-transcriptional gene silencing in animals mediated by short interfering RNAs (siRNAs) (42-47). RNAi technology may be used in accordance with the present invention to generate cells lacking a functional Sufu protein, which cells may be used in a method according to the invention, to identify inhibitors and/or antagonists of a Hedgehog signalling pathway.

“Anti-sense technology” in the context of the present invention, refers to an approach for inhibiting gene expression, particularly oncogene expression. An “antisense” RNA molecule is a molecule, which comprises the complement of, and therefore can hybridize with protein-encoding RNAs of the cell. It is believed that the hybridization of antisense RNA to its cellular RNA complement can prevent expression of the cellular RNA, perhaps by limiting its translatability. While various studies have involved the processing of RNA or direct introduction of antisense RNA oligonucleotides to cells for the inhibition of gene expression (48-51), the more common means of cellular introduction of antisense RNAs has been through the construction of recombinant vectors, which will express antisense RNA once the vector is introduced into the cell. Furthermore, DNA, synthetic oligonucleotides, PNA etc., or any other suitable molecules, may also be used in the context of Anti-sense technology. Anti-sense technology may be used in a method in accordance with the present invention, e.g. to generate cells lacking a functional Sufu protein, which may be used in a method according to the invention, to identify inhibitors and/or antagonists of a Hedgehog signalling pathway.

A “ribozyme” refers to a RNA molecule that catalytically cleaves other RNA molecules. Different kinds of ribozymes have been described in the art, including group I ribozymes, hammerhead ribozymes, hairpin ribozymes, RNAse P, and axhead ribozymes. See (52) for a general review of the properties of different ribozymes. Thus, a ribozyme may be used in a method in accordance with the present invention, e.g. to generate cells lacking a functional Sufu protein, which may be used in a method to identify inhibitors of a Hedgehog signalling pathway.

An “immunoassay” refers to an assay that utilizes an antibody to specifically bind to a substance. An immunoassay is characterised by the use of specific binding properties of a particular antibody to isolate, target and/or quantify the substance.

Immunoassays, such as “ELISA” (Enzyme Linked Immunosorbent Assay), are widely used for the determination, either qualitative or, mostly, quantitative, of a nearly unlimited variety of organic substances, either of natural origin or synthetic chemical compounds, such as peptides, proteins, enzymes, hormones, vitamins, drugs, carbohydrates, etc., for various purposes, such as in particular for diagnostic purposes, but also for forensic applications, food quality control, and generally for any analytic purpose. ELISA methods are described as comprising separate steps of incubating a sample with a first binding partner of the substance to be analysed and incubating the reaction product formed with a second binding partner of the substance. However, some existing ELISA embodiments do not comprise such separate incubation steps and allow the substance to react simultaneously, or shortly one after the other, in one and the same incubation step, with both its first and second binding partners. Competitive ELISA's are another example of ELISA variants. The present invention is in principle applicable to any and all ELISA variants, and to similar immunoassay methods which, strictly speaking, are not ELISA methods, e.g. because they do not involve the use of an enzyme. In one embodiment of the invention, an ELISA is used to detect the presence of a component of a Hedgehog signalling pathway in a cell, and/or a presence of an inhibitor and/or an antagonist of a Hedgehog signalling pathway.

A “label” or a “labelled antibody or compound” in the present context, refers to a composition detectable by spectroscopic, photochemical, biochemical, immunochemical or chemical means. Useful labels include for example ³²P, fluorescent dyes, electron-dense reagents, enzymes (e.g. as commonly used in an ELISA), biotin, dioxigenin or haptens and proteins for which antisera or monoclonal antibodies are available.

A “FACS” method, relates to fluorescence-activated cell sorting, which is a flow-cytometry analysis, enabling sorting of cell populations e.g. depending on if a specific marker is expressed or present on the cell surface. An antibody raised against this marker labelled with a fluorescent compound, is used for the detection and separation of cells with or without a marker. Many different markers may be detected at the same time. FACS techniques are well known to those skilled in the art. The use of FACS for sorting cells is discussed, for example, in U.S. Pat. No. 5,804,387. In one embodiment of the invention, FACS is used to detect the presence of a component of a Hedgehog signalling pathway in a cell using a cell surface marker.

A “cell surface marker” is a marker and/or label of any kind which is expressed on a cell's surface, and which is possible to detect by any means, such as by using FACS, or any other method which uses antibodies for detection. An example of a cell surface marker for preferable use in the context of the present invention is CD4, but it should be understood that any suitable marker may be used in the present context.

### DETAILED DESCRIPTION OF THE INVENTION

The present invention relates to a novel approach for identifying inhibitors and/or antagonists of a Hedgehog signalling pathway, a signalling pathway that, for a long time, has been known to have an implication in the development of cancer. The invention is based upon the present inventors finding that a component of the signalling pathway, Sufu, now emerges as a central and more important component of the pathway than what was initially thought. The data of the inventors show and confirm that significant differences in the mechanism of Hedgehog signalling have developed during evolution, and that Sufu has acquired a central and vertebrate-specific role in Hedgehog signal transduction.

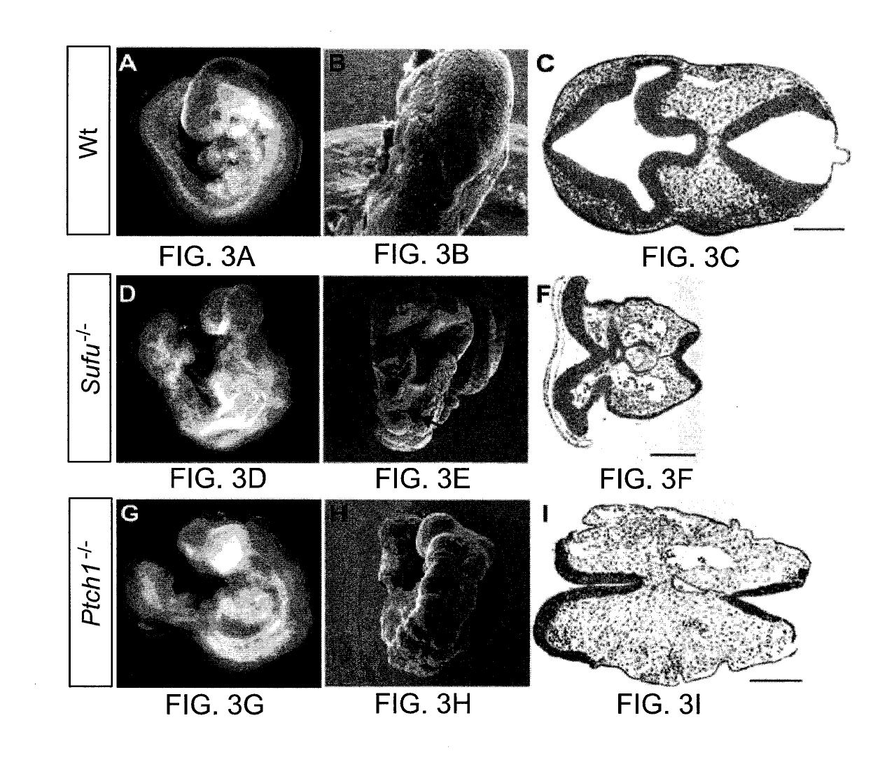

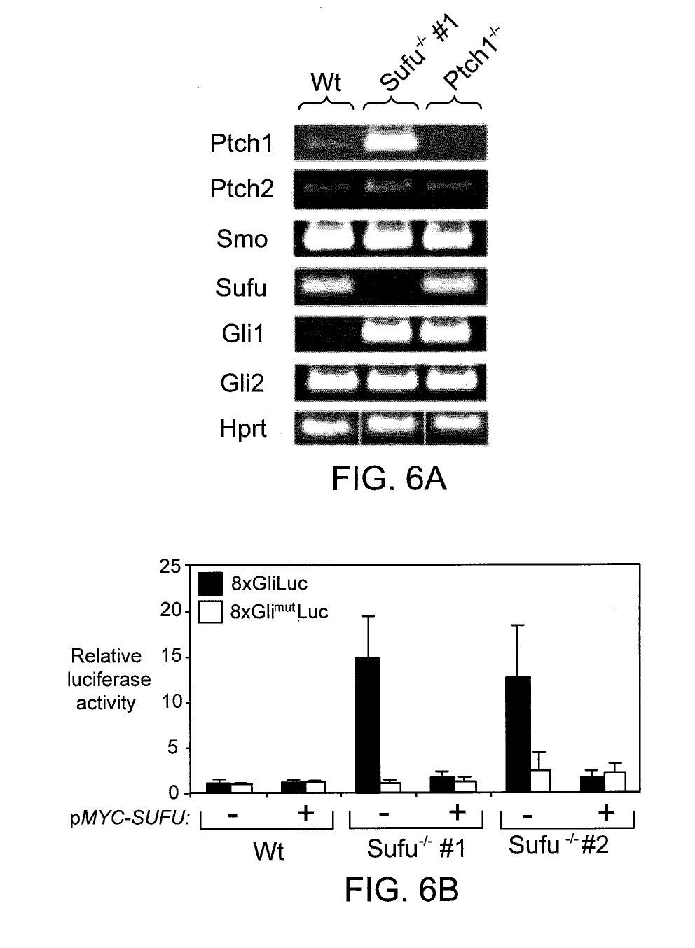

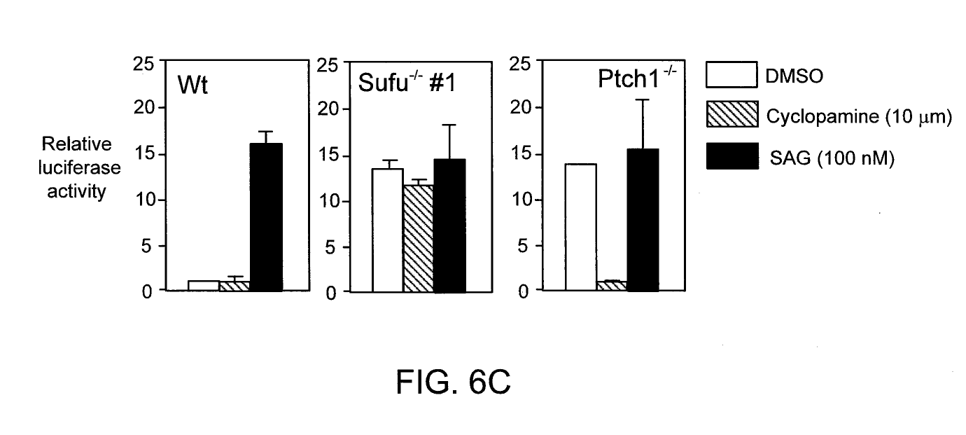

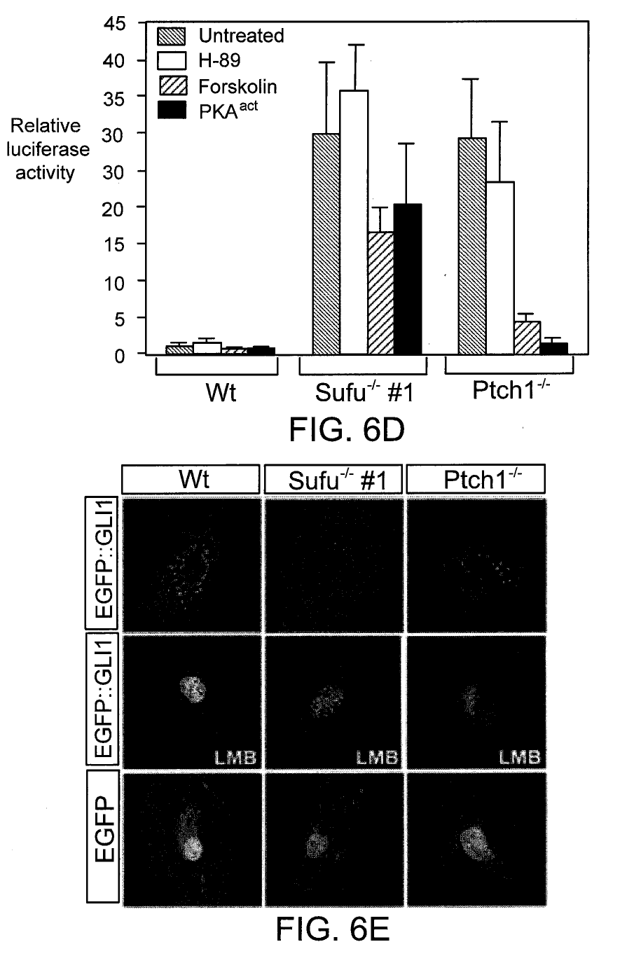

Accordingly, the inventors present an analysis of the phenotype of Sufu knockout mice. In Drosophila, the Sufu mutation was identified by its ability to suppress the effect of loss of Fused kinase (36). Sufu suppresses the effect of mutations in the kinase Fused, but has essentially no detectable phenotype when eliminated alone in the fly. Sufu interacts with the component Ci in Drosophila, and consistent with this, a vertebrate orthologue of Sufu has been identified which interacts with Gli proteins, the vertebrate counterparts of Ci (9, 33-35). Sufu appears to restrain Gli mediated transcription by inhibiting nuclear import of Glis and/or recruiting corepressors to Gli proteins (9, 53). In contrast to Drosophila, in which loss of Sufu on its own does not have an observable effect on Hedgehog signalling, the inventors show that mouse embryos lacking Sufu display a very dramatic phenotype, similar to embryos lacking Ptch1, as shown in FIG. 3, in which the absence of Sufu results in cells responding as if constitutively exposed to high levels of Hedgehog signalling. Analysis of the neural tube confirmed this, demonstrating a massive expansion of FoxA2 expression, a gene normally induced by the highest levels of Sonic Hedgehog signalling. Moreover increased Hedgehog signalling is observed in cell lines lacking Sufu, as shown in FIG. 6A.

In light of the present findings, the inventors disclose an alternative approach for a cell based assay system for identifying inhibitors and/or antagonists of a Hedgehog signalling pathway by using cells lacking a functional Sufu protein. Previously, the focus has been to screen for inhibitors, which inhibit the upstream components Smoothened (Smo) and Patched (Ptch) of the pathway. The present invention offers a way to identify an inhibitor and/or an antagonist of a Hedgehog signalling pathway, which may not be effective via the Smo/Ptch pathway, thereby providing the possibility of discovering new substances not known today, which are also able to control and inhibit a Hedgehog pathway, but which are not acting via Smo. Moreover, examples exist in the literature of cell tumors/cell lines having increased Hedgehog-signalling but lacking mutations in Ptch1 or Smo and not showing increased ligand expression. Currently available drugs (e.g. Cyclopamine, Cur61414) will not be effective in these situations, which is shown in FIG. 6. Thus, there is a need for identifying selective downstream inhibitors and/or antagonists of the Hedgehog signalling pathway which do not act via Smo. The present invention provides a method for identifying such selective downstream inhibitors and/or antagonists, and also provides proof-of concept of such an assay.

Initially, to investigate the role of the Hedgehog signalling component Sufu in mammalian systems, mouse embryonic fibroblasts (MEF) were established, in which the Sufu gene is deleted (Sufu^{-/-} MEFs). These cells recapitulate a pathological scenario in which activation of the pathway occurs through inactivating mutations of downstream negative pathway components (e.g. as was shown for some Medulloblastomas). One specifically preferred embodiment of the invention relates to said Sufu^{-/-} cells and their use in a method to identify an inhibitor and/or an antagonist of a Hedgehog signalling pathway.

The present inventors are able to show that deletion of Sufu results in full Hedgehog pathway activation, which is independent of the upstream receptors Ptch and Smo. As a consequence, existing inhibitors such as Cyclopamine, which act at the Smo level, are inactive in this setting. On the other hand, substances, e.g. inhibitors and/or antagonists, which e.g. inhibit at the Gli level instead, retain their inhibitory properties. The invention will make it possible to identify such inhibitors and/or antagonists of a Hedgehog signalling pathway, which are able to inhibit other components, being downstream of, or dependent upon, Sufu.

Due to the fact that several novel downstream components of Hedgehog signalling were recently discovered and the known occurrence of Gli gene amplification or activation by translocation, it can be expected that many more diseases than the previously mentioned Medulloblastoma are in fact caused by activating steps downstream of Smo and/or a due to lack of a functional Sufu.

The present invention enables us to, by using e.g. Sufu^{-/-} MEFs (murine embryonal fibroblasts), selectively focus on molecular targets downstream of the receptor pair Ptch/Smo. Any other cells, as considered appropriate for the particular conditions, which lack a functional Sufu protein, may also be used in a method according to the present invention. Said Sufu^{-/-} cells are encompassed by a preferred embodiment of the present invention. One way to generate Sufu^{-/-} cells in accordance with the present invention is to use two consecutive rounds of gene targeting in mouse ES cells, and/or by increasing selective pressure (commonly G418, i.e. neomycin). This presumably leads to gene conversion and the net results are two targeted alleles. Sufu^{-/-} cells could also be produced by targeting the remaining wildtype allele in Sufu^{+/-} ES cells either by using another targeting vector that utilizes another selection cassette than neo, or by culturing^{+/-} ES cells in high amount of G418 (neomycin) that will select for a gene conversion event. The invention is however not limited to the use of any of the above mentioned methods to generate Sufu^{-/-} cells. Said mouse Sufu^{-/-} embryonic fibroblasts are encompassed by a preferred embodiment of the present invention

It is to be understood that all embodiments of the present invention exclude the use of human embryonic stem cells.

The present invention proposes a method comprising using cells which lack a functional Sufu protein, such as Sufu^{-/-} cells, to discover a novel antagonist and/or inhibitor of a Hedgehog signalling pathway, targeting components downstream of Sufu, and/or downstream of Smo and Ptch, at the Gli level.

One presently preferred embodiment of the present invention comprises a cell-based assay system for identifying an inhibitor and/or an antagonist of a Hedgehog signalling pathway, comprising cells, excluding human embryonic stem cells, which lack a functional Sufu protein. In one preferred embodiment, said cells lacking a functional Sufu protein, are Sufu^{-/-} cells. In another embodiment, said cells are mammalian cells, such as mouse cells. In a presently preferred embodiment, said cells are mouse Sufu^{-/-} embryonic fibroblasts.

The present invention also relates to a method for identifying an inhibitor and/or an antagonist of a Hedgehog signalling pathway, which method comprises using a cell-based system comprising cells, excluding human embryonic stem cells, which lack a functional Sufu protein. In one embodiment, such a cell-based system may comprise Sufu^{-/-} cells. Furthermore, such a cell-based system may in another embodiment comprise mammalian cells. In one preferred embodiment of the invention, said cell-based system comprises mouse cells. In another presently preferred embodiment, said cell-based system comprises mouse Sufu^{-/-} embryonic fibroblasts. In another embodiment of the invention, a cell-based system comprises cells selected from the group consisting of prokaryotic cells, eukaryotic cells, insect cells, and yeast cells.

The present invention further relates to method for identifying an inhibitor and/or an antagonist of a Hedgehog signalling pathway, comprising the following steps: generating and/or preparing a cell-culture comprising cells lacking a functional Sufu protein, adding a substance to be tested to said cell-culture, and detecting an inhibiting and/or antagonizing function of the substance on a Hedgehog signalling pathway in said cells. In one embodiment of the invention, said cells lacking a functional Sufu protein are generated by deleting the Sufu gene from a genome, rendering Sufu^{-/-} cells.

The methods will be useful for the identification of compounds with pharmacokinetic and ADME/Tox properties useful for treatment of diseases such as cancer, keloids and granulatomous skin disorders, psoriasis, pathological neovascularization, inhibition of stem cell proliferation and induction of differentiation, excessive hair growth, body weight reduction. Substances, such as chemical compounds, which are tested for inhibiting and/or antagonizing activity herein, may be added at any preferable concentration such as between 0.1-50 μM, more preferable 0.1-10 μM and most preferable 0.1 to 1 μM.

In one specific aspect, the present invention relates to a method for identifying an inhibitor and/or an antagonist of a Hedgehog signalling pathway, comprising the following steps: generating and/or preparing a cell-culture comprising Sufu^{-/-} cells, transducing and/or transfecting said cell(s) with a reporter gene system responsive to a Hedgehog signalling gene product, adding a substances to be tested to said cells, lysing said cells, and measuring the levels of reporter gene expression and/or activity to detect an inhibiting and/or antagonizing function of the substance tested on a Hedgehog signalling pathway in said cells. In one preferred embodiment, said Sufu^{-/-} cells are generated by deleting the Sufu gene from a genome. In another embodiment, said cells may be plated onto plates with wells, which may be multiwell plates. In one embodiment, said reporter gene system comprises a plasmid for normalization. In another embodiment, said reporter gene system is responsive to Gli-mediated transcription of a gene. In yet another embodiment, said reporter gene system comprises a reporter gene comprising Gli binding sites.

In a presently preferred embodiment of the invention, said Sufu^{-/-} cells are transfected and/or transduced with a reporter gene system with 8 Gli binding sites and a phRL-SV40 plasmid or a pHRL-TK plasmid. Detection of substance activity in a method according to the invention, can typically be performed with a luciferase reporter system.

In another aspect of the invention, Sufu^{-/-} MEFs (murine embryonic fibroblast) are used to identify an antagonist and/or an inhibitor of a Hedgehog signalling pathway. In such a method, cells are treated with various substances. Cyclopamine, which acts upstream at the level of Smo, does not inhibit the pathway in the Sufu^{-/-} cells, as seen in FIG. 6. Pathway activity can in accordance with the invention be measured with a Hedgehog responsive luciferase construct (8xGli-Luc).

In yet another aspect, the invention relates to a method, which comprises the following steps: generating and/or preparing a cell culture comprising Sufu^{-/-} cells, growing untransfected cells of the genotype Sufu^{-/-} to confluency, adding a substance to be tested to said cells, preparing DNAse-treated RNA from said cells, and reversibly transcribe said RNA into cDNA, performing real-time PCR with said cDNA for the detection of Hedgehog target gene mRNA, and performing a normalization against housekeeping genes mRNA levels, using a relative and/or quantitative method detecting an inhibiting and/or antagonizing function of a substance added to said cells on a Hedgehog signalling pathway in said cells.

In one aspect of the invention, untransfected MEFs of the respective genotype (Sufu−/−; +/+) are grown to confluency, and are thereafter treated with substances to be tested for an inhibiting and/or antagonizing function at a concentration of approximately 10 μM, or any other suitable concentration. As a control, the same volume of only DMSO without test compound is added in order to assess solvent-based effects. After 3-4 days, or any other suitable amount of days, of substance treatment, DNAse-treated RNA is prepared (RNeasy Kit, Qiagen) and reverse transcribed into cDNA. Real-time PCR is performed using a commercially available kit for detection of mouse Gli1 mRNA (“Assay-on-Demand”, Applied Biosystems) on an ABI Prism 7700 (Applied Biosystems). Normalization may be performed against Gapdh mRNA levels (“Assay-on-Demand” for rodent Gapdh, Applied Biosystems) using a standard curve of a Gli1 positive sample. As a read out for Hedgehog pathway activity, Gli1 mRNA levels are measured. Only an inhibitor and/or an antagonist at the Gli level may reduce Gli1 mRNA levels. Upstream inhibitors, such as Cyclopamine, should be ineffective.

Encompassed by the present invention, is also a method, wherein said Hedgehog target gene mRNA is Gli1 mRNA. Said method may also comprise any other Hedgehog target genes for the detection of Hedgehog pathway activity downstream of Smo and Ptch. Furthermore, encompassed by the present invention is also a method wherein said normalization is performed against Gapdh mRNA levels using a standard curve of a Gli1 positive sample to detect the level of inhibition of a substance tested.

Any method disclosed herein, may be used for identifying an inhibitor and/or an antagonist of a Hedgehog signalling pathway. Any method according to the present invention may be used for identifying an inhibitor and/or an antagonist of a Hedgehog signalling pathway downstream of Smo and Ptch.

Another aspect of the present invention, relates to a method for preparing an immortalized mouse embryo fibroblast Sufu^{-/-} cell culture, which method comprises the following steps: preparing mouse embryonic Sufu^{+/-} stem cells by the introduction of a targeting vector, which vector targets a Sufu locus, into said stem cells, generating chimeric mice, intercrossing Sufu^{+/-} mice to generate Sufu^{-/-} mouse embryos, selecting Sufu^{-/-} mouse embryos, and incubating cells obtained from said Sufu^{-/-} mouse embryos in MEF medium. In one preferred embodiment, said targeting vector is pSufuΔexon1^{neo}.

According to the invention, any methods disclosed by the present invention to identify an inhibitor and/or an antagonist of a Hedgehog pathway may comprise using Sufu^{-/-} cells, prepared in accordance with a method for preparing a cell culture as disclosed herein.

The present invention also relates to a cell culture of Sufu^{-/-} cells prepared as disclosed herein. Furthermore, the invention also relates to a cell culture comprising Sufu^{-/-} cells, such as mouse embryonic fibroblasts, which cells are not human embryonic stem cells.

Enscoped by the present invention are also other means of functionally deleting or reducing the function of a Sufu gene, protein and/or transcript, within cells such as, but not limited to: RNAi technology, anti-sense technology, ribozymes, antibody injection, dominant negative variants of Sufu, blocking Sufu activating enzymes, and/or intracellular expression of antibody fragments inactivating Sufu.

Furthermore, results obtained in a method according to the invention, may be detected by; Quantitative PCR detecting mRNA for Hedgehog response genes such as Gli1, Gli2, Ptch1, Ptch2, Hip, or any other gene which belongs to the Hedgehog signalling pathway, detection of Gli1, Gli2, Ptch1, Ptch2, Hip, or any other gene which belongs to the Hedgehog signalling pathway, using antibodies, e.g in an ELISA technique, or FACS utilizing cell surface markers, or introducing a reporter gene construct and analyzing e.g. luciferase or any other reporter gene activity, such as LacZ, Alkaline Phosphatase staining, and/or fluorescent reporters such as GFP, and/or introducing a reporter gene expressing a cell surface marker such as CD4, and assaying such expression of a cell surface marker by e.g. FACS.

In one aspect of the invention, one or more cell(s) from a cell culture disclosed herein, are used for the identification of an antagonist and/or inhibitor of a Hedgehog signalling pathway. In another embodiment, said one or more cell(s) from a cell culture according to the invention, are used for the identification of an antagonist and/or inhibitor of a Hedgehog signalling pathway downstream of Smo and Ptch.

In yet another embodiment, said one or more cell(s) from a cell culture according to the invention may be used for the identification of a substance, which is able to inhibit and/or antagonize a Hedgehog signalling pathway. In yet another embodiment, said one or more cell(s) from a cell culture according to the invention may be used for the identification of a substance, which is able to inhibit and/or antagonize a Hedgehog signalling pathway downstream of Smo and Ptch. In another embodiment the invention relates to the use of a cell culture for identifying substances which induce apoptosis in a Sufu^{-/-} specific manner.

In another preferred embodiment, the invention encompasses the use of one or more cell(s) from a cell culture according to the invention for identifying a substance, which is able to selectively induce cell death by necrosis or apoptosis in a cell by inhibiting a Hedgehog signalling pathway. In another preferred embodiment, the invention encompasses the use of one or more cell(s) from a cell culture according to the invention for identifying a substance, which is able to selectively induce cell death by necrosis or apoptosis in a cell by inhibiting a Hedgehog signalling pathway downstream of Smo and Ptch. These substances are preferably not able to induce cell death in wild type cells. Such substances, are highly interesting as potential therapeutic substances, since they can potentially kill cells having a undesired property of constitutively active Hedgehog signalling.

In another aspect, the invention relates to the use of a cell-based assay system according to the invention, for identifying an inhibitor an/or an antagonist of a Hedgehog signalling pathway. In one embodiment, the cell-based assay system is used for identifying an inhibitor an/or an antagonist of a Hedgehog signalling pathway downstream of Smo and Ptch. In another preferred aspect, the present invention relates to any appropriate method disclosed herein, for identifying an inhibitor an/or an antagonist of a Hedgehog signalling pathway downstream of Smo and Ptch.

In another preferred aspect, the present invention relates to any appropriate method disclosed herein, for selecting a substance with an inhibiting and/or antagonizing function on a Hedgehog signalling pathway downstream of Smo and Ptch.

The present invention also relates to a method according to the invention to identify a substance, which inhibits and/or antagonizes a Hedgehog signalling pathway, and which may be used as a medicament for the treatment of any disease(s) and/or disorder(s) associated with a Hedgehog signalling pathway, such as, but not limited to, basal cell carcinoma, prostate cancer, breast carcinoma, and/or small-cell lung cancer, etc.

The invention also provides an animal model, which is a mouse, wherein said mouse lacks a functional Sufu protein, displaying a Sufu^{-/+} or Sufu^{-/-} phenotype. This animal model may in itself be used to identify an inhibitor and/or an antagonist of a Hedgehog signalling pathway. One embodiment of the invention thus encompasses the use of an animal model, such as a mouse, to identify an inhibitor and/or an antagonist of a Hedgehog signalling pathway. Such an animal model may of course also be used to study the effect of administering a proposed Hedgehog signalling pathway inhibitor and/or antagonist to said animal. A mouse lacking functional Sufu may be prepared in any suitable manner, such as suggested by the present invention, and which may be seen in the Experimental section (Generation of Sufu gene-targeted mice and genotyping).

### Experimental Section

### Materials and Methods

### Generation of Sufu Gene-Targeted Mice and Genotyping

The Sufu gene was isolated from the mouse 129X1/SvJ ES cell BAC library II (Genome Systems, Inc.) by hybridization screening using a 0.4 kb EcoRI fragment from a mouse Sufu EST cDNA clone (GenBank accession number AA061391). For the targeting vector, a 13.1 kb XbaI fragment containing Sufu exons 1 and 2, and a 7.7 kb EcoRI fragment containing Sufu exon 1 from the BAC clone 17985 were each subcloned into pBS II KS(+) (Stratagene). From the 13.1 kb XbaI fragment, a 3.4 kb XbaI/HindIII fragment representing the 5′ homologous arm was cloned into the XbaI and HindIII sites of a pBS II KS(+)-based plasmid vector containing the neomycin resistance cassette from pMC1-neo Poly A (54) and a HSV-tk cassette (55). To complete the targeting vector, a 1.9 kb BamHI fragment representing the 3′ homologous arm from the 7.7 kb EcoRI fragment above was subsequently cloned into the BgIII site of this vector. The final targeting vector, pSufuΔexon1^{neo}, will upon homologous recombination replace exon 1 of the Sufu gene with the neomycin cassette.

For the generation of Sufu^{+/-} embryonic stem (ES) cells, 1×10⁶ RW-4 cells from the 129X/SvJ mouse strain were transfected with 50 μg SalI-linearized pSufuΔexon1^{neo} targeting vector using a Bio-Rad Gene-Pulser with settings 230 V and 500 μF. The ES cells were cultured on γ-irradiated primary neomycin-resistant murine embryonic fibroblasts (MEFs) prepared from C57BL/6J-Tg(pPGKneobpA)3Ems/J embryos (Jackson Laboratory). ES cell culture medium consisted of KnockOut D-MEM (Invitrogen) supplemented with 15% ES-tested fetal calf serum (PAA Laboratories, GmbH, Austria), 2 mM L-glutamine (Invitrogen), 10 mM HEPES (Invitrogen), 0.1 mM MEM non essential amino acids (Invitrogen), 100 μM 2-mercaptoethanol (Invitrogen), 10 μg/ml gentamicin (Invitrogen), and 1000 units/ml ESGRO LIF (Chemicon). Positive and negative selection of ES clones was in 200 μg/ml active Geneticin (Invitrogen) and 2 μM Cymevene (Roche), respectively. Replicas of ES colonies surviving the selection was incubated at 55° C. overnight in lysis buffer containing 100 mM Tris-HCl, pH 8.5, 5 mM EDTA, 0.2% SDS, 200 mM NaCl, and 250 μg/ml proteinase K (Roche). DNA from the ES cell lysates were prepared by standard phenol/chloroform extraction followed by ethanol precipitation, digested with SacI (Promega), separated on 0.6% agarose gels and transferred to Nytran Supercharge membranes (Schleicher & Schuell) by standard Southern blotting overnight. For screening of homologous recombinants, the membranes were hybridized with a 0.4 kb EcoRI/EcoRV 3′ external probe (SEQ ID NO:30), which will detect a 6.3 kb wild-type and 17.5 kb mutant SacI fragment. The hybridization solution consisted of 5×SSC, 5×Denhardt's solution, 0.5% SDS, and 100 μg/ml denatured salmon sperm DNA (Sigma). Pre-hybridization for 4 hours and hybridization for 16 hours were performed at 65° C. followed by washing of the membrane twice 20 minutes in 2×SSC 0.1% SDS and once 20 minutes in 0.1×SSC 0.1% SDS, both washes at 65° C. The washed membranes were exposed to BioMax MS film (Kodak) with TransScreen-HE intensifying screens (Kodak) in Hypercassettes (Amersham Biosciences) at −70° C. for 1-3 days before developing.

For generation of chimeric mice, blastocysts were isolated from superovulated C57BL/6 mice (Scanbur BK, Sweden) 3.5 days after mating and injected with Sufu+/− ES cell clones. The manipulated embryos were implanted into pseudopregnant B6CBAF1 recipients (generated by in-house breeding of C57BL/6 with CBA mice from Scanbur BK, Sweden). The resulting male chimeras were backcrossed to female C57BL/6 mice (Scanbur BK, Sweden). Two out of three injected Sufu^{+/-} ES clones (6E6 and 5G11) gave germline transmission. Sufu^{+/-} mice were intercrossed to produce Sufu^{-/-} embryos. The mutant Sufu mice were provisionally designated B6;129X1/SvJ-Sufutm1Rto. Genotyping of mice and embryos was performed either by Southern as described above for the ES cell clone screening or by PCR. DNA for genotyping by Southern were from tail biopsies or whole embryos and DNA for the PCR genotyping were from ear punch biopsies or yolk sacs prepared according to the HotSHOT method (56). For the PCR genotyping, both mutant (194 bp) and wild-type (274 bp) Sufu alleles are detected in the same reaction using the primers Sufu F21 (5′-CCCTTTTTGTCAATAGTTCC-3′) (SEQ ID NO:1), Sufu R6 (5′-TGACAATAGACTCCGCCTCC-3′) (SEQ ID NO:2), and Neo F2 (5′-GCCTTCTATCGCCTTCTTGAC-3′) (SEQ ID NO:3). The 50 μl PCR reactions were run on a PTC-200 DNA engine (MJ Research) and consisted of 1×PCR Gold buffer (Applied Biosystems), 2.5 mM MgCl₂ (Applied Biosystems), 0.2 mM dNTPs (Amersham Biosciences), 0.4 μM F21 primer, 0.2 μM R6 primer, 0.2 μM Neo F2 primer (primers from CyberGene AB, Huddinge, Sweden), 1.25 units AmpliTaq Gold (Applied Biosystems), and 2 μl DNA lysate (out of 75 μl total lysate). The PCR reactions were run on 2% agarose gels, which were stained with ethidium bromide, visualized under UV-light using the Gel Doc 2000 system (Bio-Rad) and documented using the Quantity One software (Bio-Rad).

### Generation of Immortalized Sufu MEF Cell Lines

Immortalized mouse embryo fibroblast (MEF) cell lines were established using a 3T3-like protocol (57). Briefly, E9.5 embryos were incubated for 5 minutes in 0.05% trypsin-EDTA (Invitrogen) at 37° C., medium added, resuspended before plating onto tissue culture dishes, passaged until crisis and eventually immortal cells appeared. The MEF medium consisted of D-MEM (high glucose, w/o sodium pyruvate), heat-inactivated 10% fetal bovine serum (FBS), 2 mM L-glutamine, 1 mM sodium pyruvate, and 10 μg/ml gentamicin, all from Invitrogen. The Ptch1^{-/-} MEFs, derived in a similar manner, was a kind gift from Dr J. Taipale, University of Helsinki, Finland.

### Luciferase Reporter Assay

For the luciferase reporter assays, 20,000 MEF cells were seeded into each well of a 24-well plate in MEF medium. The following day, cells were transfected using FuGENE 6 (Roche) with 300 ng of the luciferase reporter plasmids 8xGliLuc and 8xGli^{mut}Luc (58) with or without 300 ng of the pMYC-SUFU expression plasmid (9), 300 ng of a constitutively active PKA catalytic subunit expression plasmid (59) or with 300 ng of the TOPflash and FOPflash reporter plasmids (Upstate Biotechnology), together with 50 ng the Renilla luciferase phRL-TK reporter (Promega) to monitor the transfection efficiency. Two days after transfection the medium was changed to low serum (0.5% FBS) with or without 10 μM cyclopamine in DMSO (Toronto Research Chemicals, Inc), 100 nM Smoothened agonist, (SAG) in methanol (provided by Dr J. Bergman, Karolinska Institutet, Sweden), 100 μM forskolin (Sigma) in DMSO, 1 μM H-89 (Sigma) in DMSO or with DMSO alone, and incubated one to three days before the cells were assayed using the Dual-Reporter Luciferase system (Promega) and the samples analyzed on a Luminoskan Ascent microplate luminometer (Thermo Electron Corp.). The assays were conducted in triplicate and repeated at least three times. The values were normalized to the Renilla reporter before calculating relative levels.

### Western Blot Analysis and ECL Detection

Total cell lysates from E9.5 embryos pooled according to genotype were prepared in extraction buffer [pH 7.6 at 4° C. containing 1% Triton X-100, 10 mM Tris-HCl, 5 mM EDTA, 50 mM NaCl, 30 mM Na-pyrophosphate, 50 mM NaF, 1 mM Na₃VO₄, 10% glycerol, and Complete protease inhibitor cocktail (Roche)]. The Triton X-100 was added after the tissue homogenization using an Ultra-Turrax T8 (IKA). Protein extracts were quantified using the DC protein assay (Bio-Rad). Fifteen μg total protein per genotype and lane in 1× Laemmlisample buffer were separated on an 8% SDS-PAGE gel using a Protean II xi Cell (Bio-Rad), and transferred to a Hybond-ECL membrane (Amersham Biosciences) using a Trans-Blot apparatus (Bio-Rad). The protein membrane was subjected to ECL analysis (Amersham Biosciences) using a primary polyclonal antibody against Sufu (1:200, sc-10933, Santa Cruz Biotechnology) and a secondary bovine α-goat IgG-HRP (1:2000, sc-2350, Santa Cruz Biotechnology). The chemiluminescent reaction was detected on Hyperfilm ECL (Amersham Biosciences). After stripping the membrane, protein loading was verified using a primary monoclonal α-actin antibody (1:5000, A5441, Sigma) and a secondary sheep α-mouse IgG-HRP (1:5000, NA-931, Amersham Biosciences).

### Northern Blot, RT-PCR and Real-Time Quantitative PCR

For the Northern analysis, total RNA from E8.5 and E9.5 embryos were prepared using the RNeasy kit (Qiagen). Embryos were collected in RNAlater (Ambion) prior to preparation. For the Northern gel, 15 μg total RNA per lane was separated on a 1% formaldehyde-containing agarose gel, and transferred to Nytran Supercharge membranes (Schleicher & Schuell) in NorthernMax transfer buffer (Ambion) followed by UV-crosslinking. The radioactively labeled probes consisted of a 1.2-kb mouse Sufu cDNA XhoI/NotI fragment from an EST clone (GenBank acc. number AA754906) and a 1076-bp mouse β-actin cDNA fragment (Ambion). Pre-hybridizations were for 1-2 hours and hybridizations were for 2-3 hours at 65° C. in ULTRAhyb (Ambion) followed by three washes at 65° C. in Low Stringency buffer 1 (Ambion) and two washes at 65° C. in High Stringency buffer 2 (Ambion). For the RT-PCR analysis, total RNA from confluent MEF cells or embryos (Actr1a analysis) were prepared as above with RNase-free DNasel (Qiagen) treatment and reverse transcribed into cDNA using SuperScript (Invitrogen). PCR amplifications were performed with primers against mouse Ptch1, Ptch2, Smo, Sufu, Gli1, Gli2, Actr1a, Hprt, and Actr1a (Table 1).

TABLE 1
Primers used in this study.

| | | | Product | |
|---|---|---|---|---|
| Gene | 5′ primer | 3′ primer | length | Ref |
| Sufu wt^{a} | F21: | R6: | 274 bp | (60) |
| | 5′-CCCTTTTTGTCAATAGTTCC-3′ | 5′-TGACAATAGACTCCGCCTCC-3′ | | |
| | (SEQ ID NO:4) | (SEQ ID NO:5) | | |
| Sufu mut^{a} | F21: | Neo F2: | 194 bp | (60) |
| | 5′-CCCTTTTTGTCAATAGTTCC-3′ | 5′-GCCTTCTATCGCCTTCTTGAC-3′ | | |
| | (SEQ ID NO:6) | (SEQ ID NO:7) | | |
| Sufu mut^{a} | TKp: | R33: | 2.3 kb | (60) |
| | 5′-GCAAAACCACACTGCTCGAC-3′ | 5′-TTCTCCCCCAACTTCTGCTGCCAATCTCC-3′ | | |
| | (SEQ ID NO:8) | (SEQ ID NO:9) | | |
| Ptch1 wt^{a} | F2 | R2 | 536 bp | (60) |
| | 5′-AGTATGGCTCATTGGTTCTTGGG-3′ | 5′-CTCCCCTTGCCTGGTCTGTGTGT-3′ | | |
| | (SEQ ID NO:10) | (SEQ ID NO:11) | | |
| lacZ(Ptch1 mut)^{a} | 5′-TTCACTGGCCGTCGTTTTACAACGTCGTGA-3′ | 5′-ATGTGAGCGAGTAACAACCCGTCGGATTCT-3′ | 364 bp | (61) |
| | (SEQ ID NO:12) | (SEQ ID NO:13) | | |
| Ptch1^{b} | 5′-GACCGGGACTATCTGCA-3′ | 5′-CTCCTATCTTCTGACGGGT-3′ | 682 bp | (60) |
| | (SEQ ID NO:14) | (SEQ ID NO:15) | | |
| Ptch2^{b} | 5′-TCCAAGGTCTACTCTTCTCC-3′ | 5′-GCTCCTCGAGCAGCTGCTGA-3′ | 555 bp | (62) |
| | (SEQ ID NO:16) | (SEQ ID NO:17) | | |
| Smo^{b} | 5′-TGGGATCCAGTGCCAGAACCCGCT-3′ | 5′-ACGGTACCGATAGTTCTTGTAGCC-3′ | 562 bp | (62) |
| | (SEQ ID NO:18) | (SEQ ID NO:19) | | |
| Sufu^{b} | 5′-CTCCAGGTTACCGCTATCGTC-3′ | 5′-CACTTGGTCCGTCTGTTCCTG-3′ | 190 bp | (60) |
| | (SEQ ID NO:20) | (SEQ ID NO:21) | | |
| Gli1^{b} | 5′-TTCGTGTGCCATTGGGGAGG-3′ | 5′-CTTGGGCTCCACTGTGGAGA-3′ | 444 bp | (62) |
| | (SEQ ID NO:22) | (SEQ ID NO:23) | | |
| Gli2^{b} | 5′-TTCGTGTGCCGCTGGCAGGC-3′ | 5′-TTGAGCAGTGGAGCACGGAC-3′ | 425 bp | (62) |
| | (SEQ ID NO:24) | (SEQ ID NO:25) | | |
| Hprt^{b} | 5′-TACAGGCCAGACTTTGTTGG-3′ | 5′-AACTTGCGCTCATCTTAGGC-3′ | 152 bp | (63) |
| | (SEQ ID NO:26) | (SEQ ID NO:27) | | |
| Actr1a^{b} | F1: | R1: | 310 bp | (60) |
| | 5′-GACCGGGCTGGCAGTTCCTTC-3′ | 5′-TGCGTTCCATGTCGTTCCAGTCC-3′ | | |
| | (SEQ ID NO:28) | (SEQ ID NO:29) | | |

^{a}Primer pairs used in genomic PCR
^{b}Primer pairs used in RT-PCR

For the real-time quantitative PCR, total RNA from fresh ˜1-year-old Sufu^{+/+} and Sufu^{+/-} skin tissue on B6 congenic background (N7) or frozen ˜2-year-old Sufu^{+/+} and Sufu^{+/-} skin tissue on mixed B6;129 background were prepared (64). The RNA samples were treated with RNase-Free DNasel (Promega) and one μg reverse transcribed using SuperScript (Invitrogen) with oligo(dT)₁₅ primers. The reactions were made up in TaqMan Universal PCR master mix (Applied Biosystems) with TaqMan Gene Expression Assays for mouse Gli1 (assay ID #Mm00494645_m1) and endogenous control mouse GAPDH (#4352339E) and analyzed on an ABI PRISM 7700 Sequence Detection System (Applied Biosystems). Samples were analyzed in triplicates for each dilution and normalized to GAPDH in each sample before calculating relative Gli1 mRNA levels.

### Scanning Electron Microscopy, In Situ Hybridizations, and Histochemistry

For the scanning electron microscopy, embryos were fixed in 2% glutaraldehyde, dehydrated in ascending concentrations of ethanol, dried by critical point and mounted on aluminum stubs. After coating with 15 nm platinum, the samples were analyzed in a Jeol JSM-820 scanning electron microscope operating at 15 kV. Whole-mount in situ hybridization was performed on E8.5 and E9.5 paraformaldehyde-fixed embryos (65). Mouse antisense and sense (control) RNA probes were prepared using DIG RNA labeling mix (Roche) together with T3, T7, or SP6 RNA polymerases (Roche). The linearized plasmids used as templates for the in vitro transcription were mouse cDNA fragments for Shh (0.6 kb in pBS II KS+), Ptch1 [841 bp in pBS II KS+; (66)], Sufu [987 bp in pBS SK−; (67)], Gli1 (552 bp in pBS II KS+), Gli2 (1.0 kb in pBS II KS+), Gli3 (1.1 kb in pBS II SK+), and Axin2/Conductin [637 bp in pBS II SK+; (68)]. The Shh-hybridized embryos were paraffin-embedded and sectioned. Immunohistochemical localization of proteins in the neural tube was performed as described (69, 70). Antibodies against Shh, Nkx2.2, FoxA2 and Pax6 were obtained from the Developmental Studies Hybridoma Bank. The Nkx6.1 (71), and Isl1/2 antibodies (72) have been described. NK1R antisera (s8305, Sigma) was a kind gift from Dr G. Fortin, France. Images were collected using a Zeiss LSM510 confocal microscope.

In situ hybridization of neural tube sections was performed as described (73) using DIG-labeled cRNA probes for mouse Ptch1, Gli3 and Nato3 (74). For the histological analysis, embryos and adult mouse tissue were fixed in 10% neutral-buffered formalin (Sigma) or Bouin's solution (Sigma), and subsequently paraffin embedded, sectioned at 4-5 μm and stained with hematoxylin-eosin or followed by immunohistochemistry.

### GLI1 Subcellular Localization in MEFs

Wt, Sufu^{-/-} and Ptch1^{-/-} MEFs (1×10⁶ cells) were each transfected with 5 μg of the expression constructs containing GLI1 cDNA fused in frame 3′ of EGFP [EGFP::GLI1, (9)] or with the parental EGFP plasmid alone (BD Biosciences Clontech) in MEF 1 solution using Nucleofector technology, program T-20 (Amaxa Biosystems), and seeded onto slide chambers followed by culturing overnight. Twenty-four hours after transfection, cells were either treated with 10 ng/ml Leptomycin B (Sigma) for 3-6 hours or left untreated, washed in PBS, fixed in 4% PFA, washed in PBS again and incubated with DRAQ5 (Alexis Biochemicals) for visualization of cell nuclei, followed by mounting using ProLong Gold antifade reagent (Invitrogen). Fluorescence was visualized using a Zeiss LSM510 confocal microscope with lasers set at 488 nm for EGFP and at 633 nm for DRAQ5.

### Plasmids

The Hh reporter plasmids 12xGliBS-Luc and 8xGli-Luc are described in (9) and (58), respectively. The Renilla luciferase plasmid and the constitutive expression plasmid for β-Galactosidase were obtained from Promega (phRL-TK, pSV-βGal). The GLI1 expression plasmid are described in (9). GLI2 (ΔN-GLI2, GLI2β) expression plasmid was obtained from G. Regl (75). Transfections were done using Fugene (Roche) according to instructions given by the manufacturer.

### Substance Testing

For the substance testing, Sufu−/− MEF cells are transfected with 8xGli and phRL-SV40 (or phRL-TK) plasmids using Fugene transfection reagent on larger plates. The day after transfection, cells are replated on suitable microwell plates. When cells reach confluency, substances, which are tested for inhibiting activity, (dissolved in DMSO) are added at a concentration of approximately 10 μM, or any other appropriate concentration. As control, the same volume of only DMSO without test compound is added (reaching a final concentration of e.g. 0.5-2% of DMSO in the cell culture medium) in order to assess solvent-based effects. Cells are lysed approximately 3-4 days later, and luciferase levels are measured using the Dual-Reporter Luciferase system (Promega) or any other suitable method.

### Hh Reporter Assays

Hh reporter assays were performed essentially as described (59, 60). Signals were recorded using the Dual Luciferase Kit (Promega) for luciferase measurements and Galacto-Light Plus (Applied Biosystems) for β-Galactosidase measurements.

### Results

### Sufu^{-/-} Embryos Die in Utero at ˜E9.5

To gain insight into the function of Sufu in mammals, homologous recombination in mouse ESCs was used to generate mice with a functional ablation of the Sufu gene (see Supplemental data). Unexpectedly, embryos homozygous for the Sufu null allele die in utero around E9.5 with a severely deformed cephalic region that includes an open fore-, mid-, and hindbrain and neural tube (FIGS. 3D-3F). Compared to other mouse Hh pathway loss-of-function mutants, there is a striking similarity with Ptch1^{-/-} embryos, which die around the same age with similar morphology [(76) and FIGS. 3G-3I].

### Constitutive Activation of the Hh Pathway in the Sufu^{-/-} Embryos

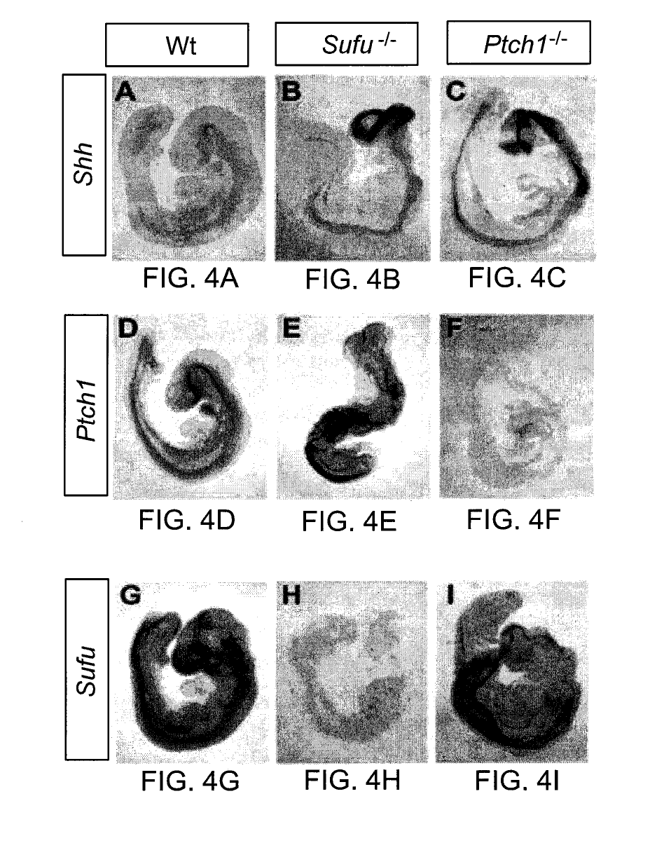

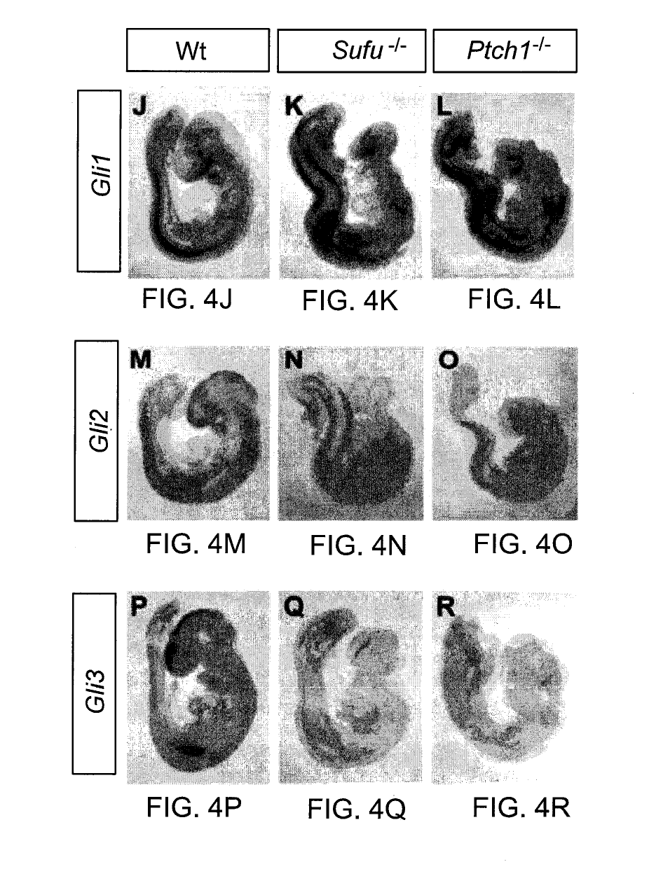

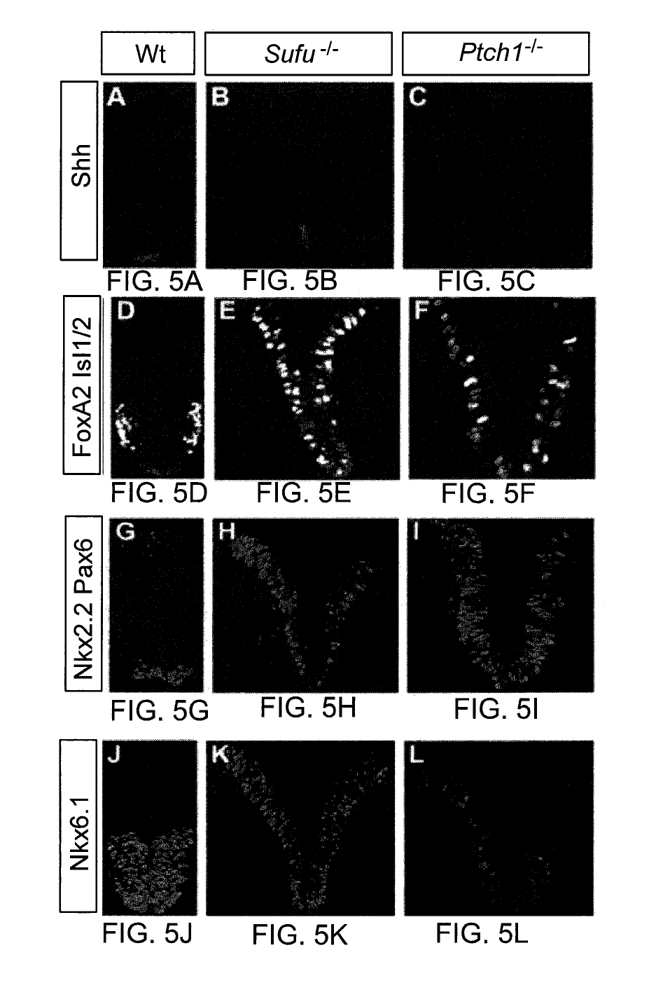

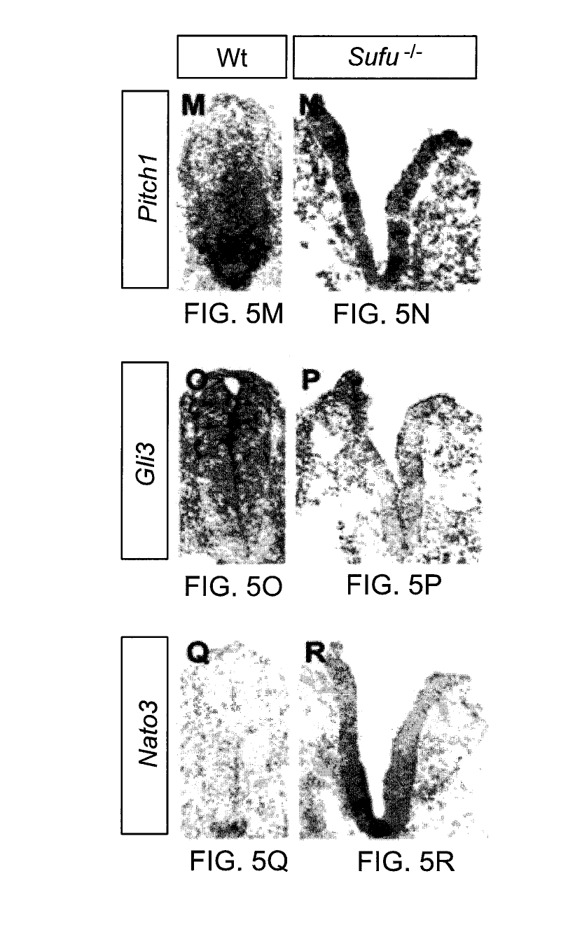

To investigate whether global changes in expression of Hh pathway components can be found in the Sufu^{-/-} embryos, we used in situ hybridization on whole-mounts and sections in E8.5 (data not shown) and E9.5 embryos (FIG. 4). Strikingly, there was a significant change in the expression pattern of both Ptch1 and Gli1, particularly along the entire neural tube where the expression domain was much broader and extended dorsally (FIGS. 4E, 4K, and 5N) compared to wild-type (Wt) embryos (FIGS. 4D, 4J, and 5M). Moreover, the Gli1 expression pattern was very similar in both Sufu^{-/-} and Ptch1^{-/-} embryos (FIGS. 4K and 4L). The expression of both Gli2 and Gli3 in the cephalic region was absent the Sufu^{-/-} embryos, consistent with the fact that the cephalic neural folds did not close (FIGS. 4N and 4Q). Besides the cephalic region, in the rest of the embryo including the neural tube, there was a marked lower overall expression of Gli3 (FIGS. 4Q and 5P). In contrast, particularly in the caudal region, Gli2 expression remained detectable (FIG. 4N). Sufu expression is normally rather widespread (FIG. 4G) and no major changes occur in the Ptch1^{-/-} embryos (FIG. 41) suggesting that Sufu is not itself a transcriptionally regulated Hh target gene.

### Sufu^{-/-} Embryos Develop a Ventralized Neural Tube

The role of Hh signalling activity in patterning of the dorso-ventral aspects of the developing neural tube is well characterized (77). Shh secreted from the notochord and floor plate forms a dorso-ventral gradient that translates into a gradient of Gli activity. This in turn controls cell fate and position of the different neuronal subtypes of the ventral neural tube (78). In Ptch1^{-/-} embryos, the neural tube is ventralized (76). To gain insight into how loss of Sufu affects levels of Hh signalling, we investigated possible qualitative and/or quantitative differences in the patterning of the E9.5 Sufu^{-/-} neural tube compared to Ptch1^{-/-} and Wt embryos. FoxA2 (Hnf3β), a winged-helix transcription factor, is important for floor plate (FP) development in the neural tube and is also a marker, albeit not a definitive one, for FP cells [(79) and FIG. 5D]. In the Sufu^{-/-} neural tube, FoxA2 was expressed along the entire dorso-ventral axis (FIG. 5E), suggesting that, like in Ptch1^{-/-} embryos (FIG. 5F), the neuroepithelium has adopted a ventralized identity. However, as shown by using the more definitive FP marker NK1R, a substance P receptor (data not shown) and Nato3 (FIG. 5R) not all cells in the neural tube were bona fide FP cells. The FP cells normally express Shh [(79) and FIG. 4A], regulated by a FP-specific enhancer in the Shh gene, which contains FoxA binding sites (80). Indeed, the expanded FoxA2 domain corresponded to the dorsal expansion of Shh protein and mRNA expression (FIG. 5B) To understand whether the different neuronal subtypes were forming in the Sufu^{-/-} neural tube, we immunostained for the Nkx2.2 and Nkx6.1 homedomain proteins, both of which were mis-expressed along the entire dorso-ventral axis (FIGS. 5H and 5K) compared to Wt (FIGS. 5G and 5J) mirroring the expression seen in the Ptch1^{-/-} neural tube (FIGS. 5I and 5L). The homedomain protein Isl1/2 is normally expressed in the motor neuron (MN) domain and the number of Isl1/2 immunoreactive cells in the Sufu^{-/-} (FIG. 5E) and Ptch1^{-/-} neural tube (FIG. 5F) appear rather similar to Wt (FIG. 5D). However, in both mutants, the Isl1/2-positive cells are scattered along the dorso-ventral axis rather than confined to the discrete MN domain. In contrast, the homedomain protein Pax6, normally expressed in the dorsal neural tube was essentially lost in both Sufu^{-/-} (FIG. 5H) and Ptch1^{-/-} (FIG. 5I) compared to Wt (FIG. 5G). However, in the region of neural tube closure, Ptch1^{-/-} mutants display Pax6 expression at the most dorsal region (data not shown) as has been described before (81). These data demonstrate that the Sufu^{-/-} neural tube shows a strong ventralization with most neuronal cells committed to a ventral fate.

### The Constitutive Gli Activity in the Sufu^{-/-} MEFs is Unaffected by Either a Smoothened Agonist or Antagonist and is Partially Sensitive to PKA Inhibition

To further explore the Sufu-dependent Hh signalling defects in more detail, we analyzed embryonic fibroblast (MEF) cell lines established from Sufu^{+/+} (Wt), Sufu^{+/-}, and Sufu^{-/-} E9.5 embryos with the same genetic background. Ptch1^{-/-} MEFs were used in comparison (59). The expression of the Hh target genes Ptch1, Gli1, and to some extent Ptch2, were upregulated in the Sufu^{-/-} MEFs compared with the Wt MEFs as shown by RT-PCR (FIG. 6A) consistent with the constitutively active Hh signalling seen in the embryos. A similar expression pattern was seen in the Ptch1^{-/-} MEFs. Gli2 and Smo expression remained unchanged in the Sufu^{-/-} as well as the Ptch1^{-/-} MEFs compared to Wt. The absence of Sufu mRNA in the Sufu^{-/-} MEFs was confirmed and the expression of Sufu was not altered in the Ptch1^{-/-} MEFs (FIG. 6A). To assess the activity of the Gli transcription factors in the MEFs, we used a luciferase assay with reporter plasmids containing eight Wt (8xGliLuc) or mutant (8xGli^{mut}Luc) Gli binding sites in tandem (58). Both Sufu^{-/-} MEF lines #1 and #2 showed a similar ˜12-15-fold increase in reporter activity relative to Wt MEFs (FIG. 6B). This increased activity is dependent on intact Gli binding sites since the 8xGli^{mut}Luc reporter gave essentially no significant activity above that found in Wt MEFs (FIG. 6B).

We next asked if we could rescue the phenotype of the Sufu^{-/-} MEF cells and repress the increased Gli activity by re-introducing Sufu. Transient transfection of human Sufu caused a reduction in Gli activity to Wt levels (FIG. 6B). This demonstrates that the observed phenotype is Sufu-dependent and not due to other genetic alterations in the ES cells or MEF cells. Furthermore, it shows that human Sufu can functionally substitute for mouse Sufu. To address the question whether the observed Gli-mediated Hh signalling activity in the Sufu^{-/-} MEF cells can be further stimulated, we incubated the Sufu^{-/-} MEF cells with or without a concentration of Smo agonist (SAG) that has been shown to fully activate the Hh pathway (82). No further increase in Gli reporter activity could be observed (FIG. 6C) suggesting that in the absence of Sufu, activation of Smo could not induce additional Gli-mediated transcriptional activity. Conversely, we tested whether cyclopamine, a known inhibitor of the Hh pathway that acts on Smo, could inhibit Gli reporter activity (59). However, again no effect was observed (FIG. 6C), indicating that neither stimulation nor inhibition of the Hh pathway at the level of Smo has any significant effect on Gli activity in the absence of Sufu.

Protein kinase A (PKA) is known to negatively affect Hh signalling. To test whether PKA has such an effect in cells lacking Sufu, we treated MEF cells with forskolin, a known activator of PKA, and could inhibit the Gli-mediated response but only by approximately 50% (FIG. 6D). The same result was obtained by expressing a constitutively active form of the catalytic subunit of PKA. In contrast, an almost complete suppression was observed in the Ptch1^{-/-} MEFs (FIG. 6D), as has been shown before (59). An inhibitor of PKA, H-89, as expected had no significant effect on the Gli response (FIG. 6D).

### EGFP:Gli1 Localize Predominantly in the Cytoplasm of Sufu^{-/-} MEFs

One of the proposed roles for Sufu is to retain Gli:s in the cytoplasm. To determine the subcellular localization of Gli1 in the absence of Sufu, we transiently transfected the MEF cells with Gli1 fused to EGFP (EGFP::Gli1) to visualize the protein. EGFP::Gli1 localized predominantly in the cytoplasm of Sufu^{-/-} MEFs and no significant difference to Wt and Ptch1^{-/-} MEFs was observed (FIG. 6E). Transfection of the parental EGFP plasmid gave a different localization pattern, which was both cytoplasmic and nuclear (FIG. 6E), indicating that the localization we observed is Gli1-specific and not a property of the EGFP protein. The nuclear export of Gli:s is dependent on Crm1 (9), which facilitates the translocation of proteins with a nuclear export signal, a process that can be blocked by Leptomycin B (LMB). To investigate whether the cytoplasmic localization of Gli1 in the Sufu^{-/-} MEFs is dependent on active nuclear export, we treated EGFP::Gli1-transfected MEFs with LMB and observed a strong nuclear retention of the EGFP::Gli1 fusion protein regardless of MEF genotype (FIG. 6E). The control EGFP subcellular localization remained unchanged upon LMB treatment (data not shown).

### Sufu^{+/-} Mice Develop a Skin Phenotype with Gorlin-Like Features

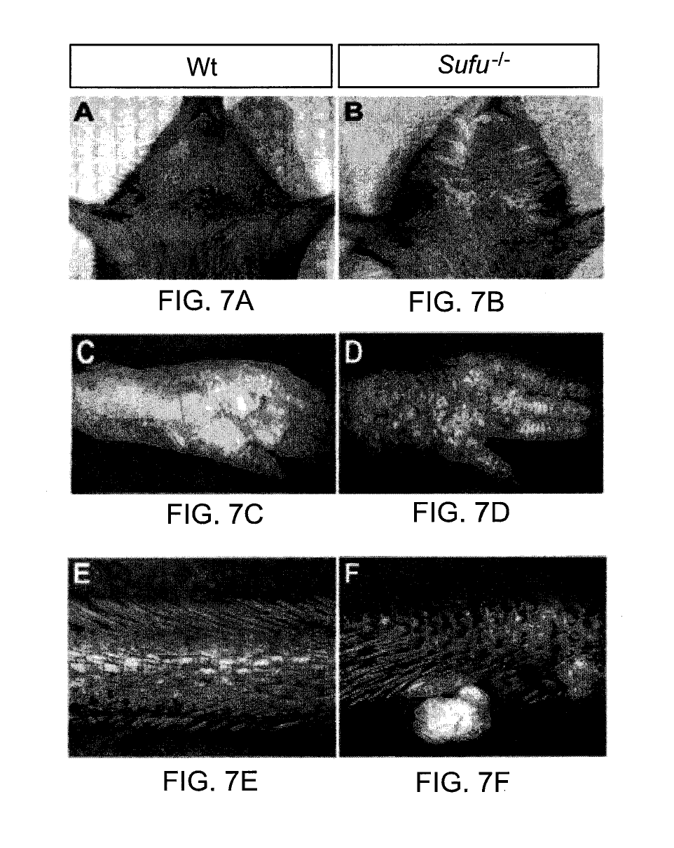

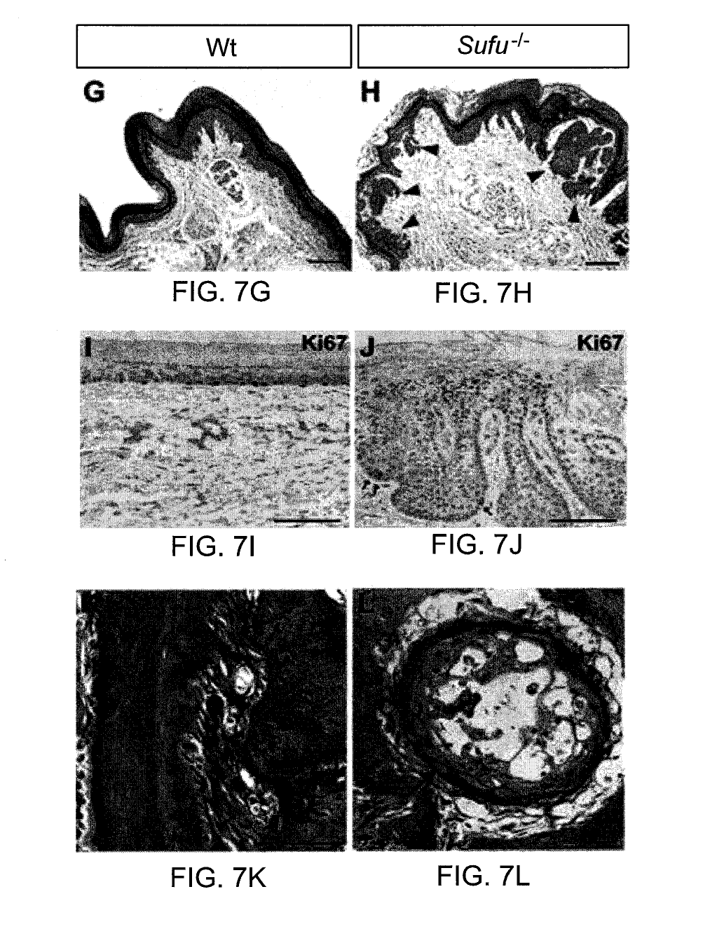

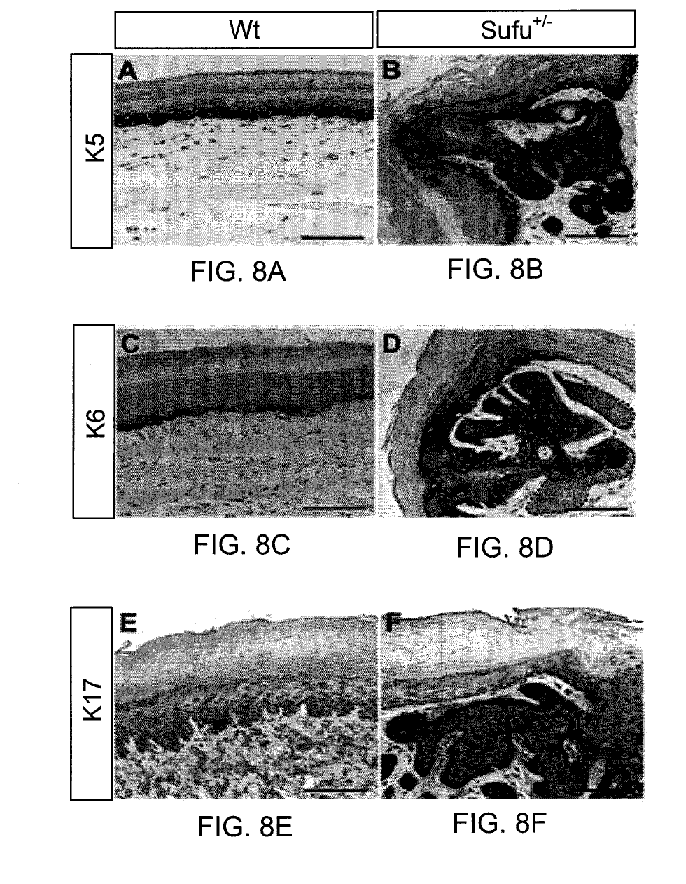

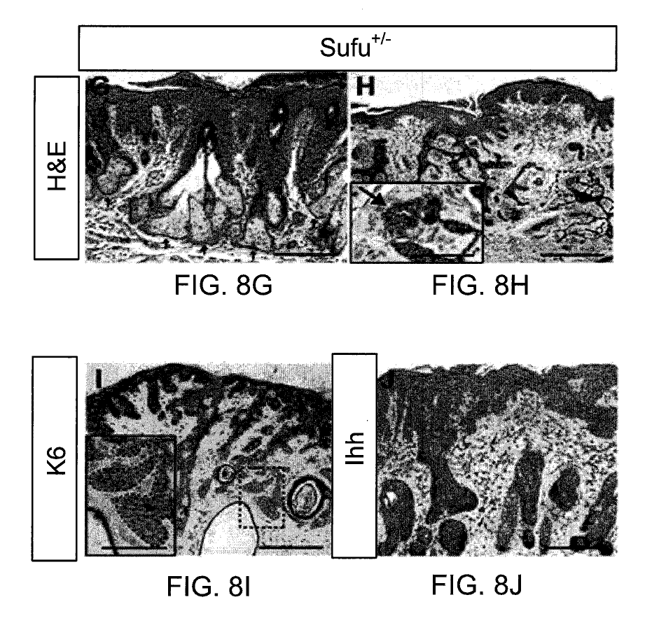

Having established that homozygous inactivation of Sufu in mouse embryos and MEFs results in constitutive and seemingly full activation of the Hh signalling pathway, we asked the question if any Hh-related phenotypic changes are present in Sufu^{+/-} mice. Such mice are born at the expected Mendelian ratio, appear normal at birth, show normal growth, and are fully fertile. However, a distinct skin phenotype with 100% penetrance (43/43 mice examined) developed in the Sufu^{+/-} mice, macroscopically characterized by ventral alopecia (FIG. 7B), increased pigmentation (FIGS. 7B, 7D and 7F), and papules and nodules on the paws and tail (FIGS. 7D and 7F) macroscopically visible from ˜1.5 years of age and becoming more severe in older mice. The earliest microscopic alterations were seen as small basaloid evaginations arising from the basal epidermal cells on the palmar aspect of the paws, at about six months of age. By two years of age, alterations were found in all skin areas (FIG. 7H). Immunohistochemical staining for the proliferation marker Ki67 revealed a relatively low number of positive cells (FIG. 7J) consistent with the observed slow growth of these changes. Additional characterization of the skin phenotype can be found in the Supplemental data. Furthermore, we frequently observed the appearance of mandibular keratocysts in the Sufu^{+/-} mice (FIG. 7L), which is a typical finding in Gorlin patients.

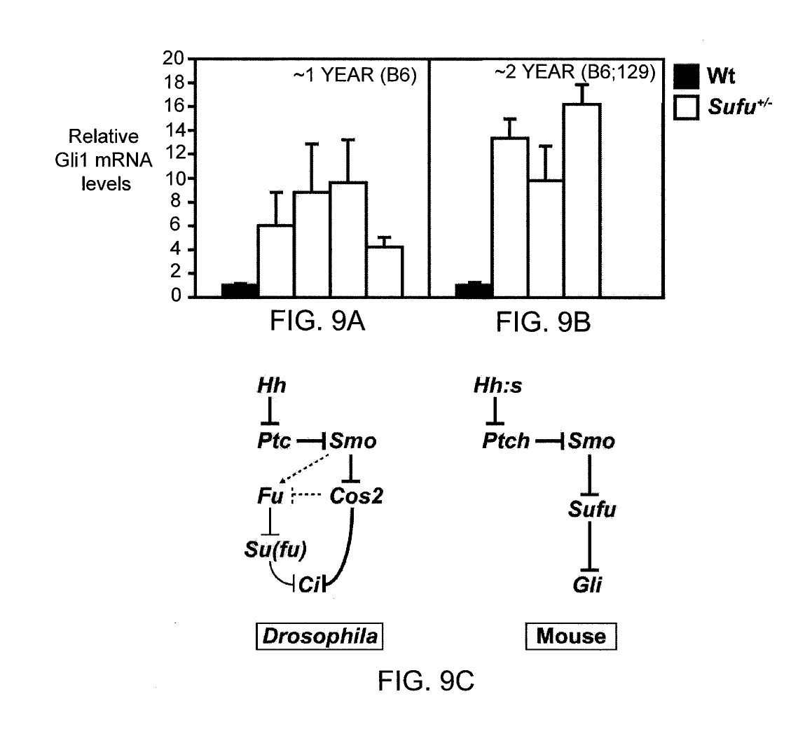

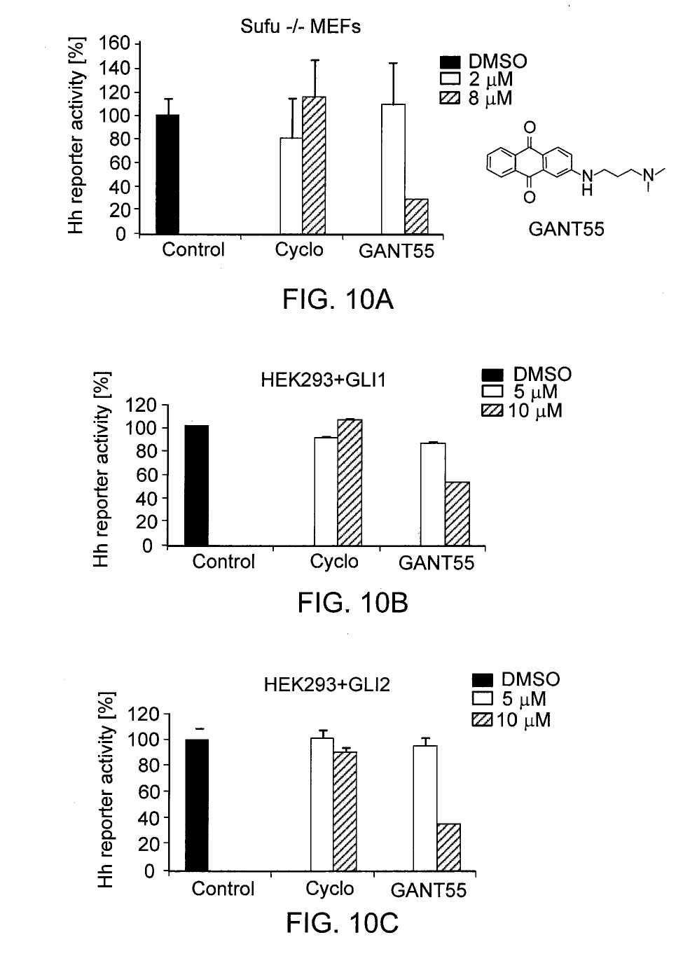

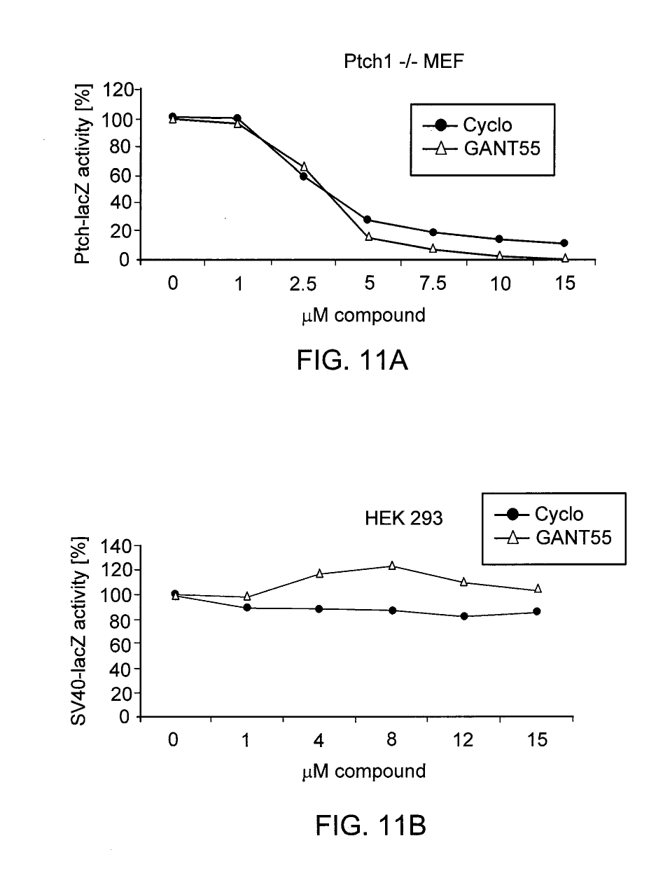

Overexpression of the Hh-effectors Gli1 and Gli2 (10, 83) as well as a constitutively active form of Smo (84) in mouse skin drives the development of a range of hair follicle-associated lesions with phenotypes correlating to different levels of Gli expression. To investigate whether increased Hh signalling is underlying the appearance of skin lesions in Sufu^{+/-} mice, we measured the levels of Gli1 expression by quantitative real-time PCR in the skin of ˜2-year-old Sufu^{+/-} and Wt mice on a mixed B6;129 background. Three out of three tested Sufu^{+/-} mice showed a 9.8-16.2-fold increase in Gli1 mRNA levels compared to age-matched Wt control (FIG. 9B). Moreover, in younger mice with less advanced proliferations, ˜1-year old on a B6 congenic background (N7), smaller but still significant increases (4.2-9.6-fold) were found in four out of four Sufu^{+/-} mice compared to Wt control (FIG. 9A). Thus, the increase in Gli1 expression levels correlate with extent of the epidermal skin changes. A preliminary comparison of skin manifestations in heterozygous Ptch1 and Sufu mice on the same genetic background reveal that the skin phenotype is tied to Sufu haploinsufficiency; very few Ptch1^{+/-} mice developed these skin manifestations.

### REFERENCES

1. P. A. Lawrence, G. Struhl, Cell 85, 951 (1996).
2. L. Lum, P. A. Beachy, Science 304, 1755 (2004).
3. P. W. Ingham, A. P. McMahon, Genes Dev 15, 3059 (2001).
4. J. Taipale, P. A. Beachy, Nature 411, 349 (2001).
5. S. S. Karhadkar et al., Nature 431, 707 (2004).
6. D. N. Watkins et al., Nature 422, 313 (2003).
7. S. P. Thayer et al., Nature 425, 851 (2003).
8. D. M. Berman et al., Nature 425, 846 (2003).
9. P. Kogerman et al., Nat Cell Biol 1, 312 (1999).
10. M. Nilsson et al., Proc Natl Acad Sci USA 97, 3438 (2000).
11. S. H. Shin, P. Kogerman, E. Lindstrom, R. Toftgard, L. G. Biesecker, Proc Natl Acad Sci USA 96, 2880 (1999).
12. K. W. Kinzler, B. Vogelstein, Mol Cell Biol 10, 634 (1990).
13. N. Dahmane, J. Lee, P. Robins, P. Heller, A. Ruiz i Altaba, Nature 389, 876 (1997).
14. A. Ruiz i Altaba, Development 126, 3205 (1999).
15. P. Dai et al., J Biol Chem 274, 8143 (1999).
16. H. Hahn et al., Cell 85, 841 (1996).
17. R. L. Johnson et al., Science 272, 1668 (1996).
18. A. Vortkamp, M. Gessler, K. H. Grzeschik, Nature 352, 539 (1991).
19. R. Toftgard, Cell Mol Life Sci 57, 1720 (2000).
20. P. Sanchez et al., Proc Natl Acad Sci USA 101, 12561 (2004).
21. U. Tostar et al., J Pathol 208, 17 (2006).
22. X. Ma et al., Int J Cancer 118, 139 (2006).
23. X. Ma et al., Carcinogenesis 26, 1698 (2005).
24. J. K. Sicklick et al., Carcinogenesis, (2005).
25. A. Dahlen et al., Am J Pathol 164, 1645 (2004).
26. M. Katano, Cancer Lett 227, 99 (2005).
27. A. Ruiz i Altaba, B. Stecca, P. Sanchez, Cancer Lett 204, 145 (2004).
28. G. Regl et al., Cancer Res 64, 7724 (2004).
29. T. Tsuji, L. Catasus, J. Prat, Hum Pathol 36, 792 (2005).
30. B. Stecca, A. Ruiz i Altaba, J Neurobiol 64, 476 (2005).
31. R. D. Paladini, J. Saleh, C. Qian, G. X. Xu, L. L. Rubin, J Invest Dermatol 125, 638 (2005).
32. L. Milenkovic, L. V. Goodrich, K. M. Higgins, M. P. Scott, Development 126, 4431 (1999).
33. Q. Ding et al., Curr Biol 9, 1119 (1999).
34. M. Murone et al., Nat Cell Biol 2, 310 (2000).
35. R. V. Pearse, 2nd, L. S. Collier, M. P. Scott, C. J. Tabin, Dev Biol 212, 323 (1999).
36. T. Preat, Genetics 132, 725 (1992).
37. T. Preat et al., Genetics 135, 1047 (1993).
38. J. A. Williams et al., Proc Natl Acad Sci USA 100, 4616 (2003).
39. J. Sambrook, D. W. Russell, Molecular cloning: A laboratory manual. (Cold Spring Harbor Laboratory Press, Cold Spring Harbor, ed. 3rd, 2001).
40. F. M. Ausubel et al., Eds., Current protocols in molecular biology, (John Wiley & Sons, New York, 1998).
41. R. A. Woo, R. Y. Poon, Genes Dev 18, 1317 (2004).
42. P. D. Zamore, T. Tuschl, P. A. Sharp, D. P. Bartel, Cell 101, 25 (2000).
43. A. Fire et al., Nature 391, 806 (1998).
44. A. J. Hamilton, D. C. Baulcombe, Science 286, 950 (1999).
45. R. Lin, L. Avery, Nature 402, 128 (1999).
46. P. A. Sharp, Genes Dev 13, 139 (1999).
47. E. Strauss, Science 286, 886 (1999).
48. D. Brown et al., Oncogene Res 4, 243 (1989).
49. E. L. Wickstrom et al., Proc Natl Acad Sci USA 85, 1028 (1988).
50. C. C. Smith, L. Aurelian, M. P. Reddy, P. S. Miller, P. O. Ts'o, Proc Natl Acad Sci USA 83, 2787 (1986).
51. M. Buvoli, G. Biamonti, S. Riva, C. Morandi, Nucleic Acids Res 15, 9091 (1987).
52. D. Castanotto, J. J. Rossi, N. Sarver, Adv Pharmacol 25, 289 (1994).
53. S. Y. Cheng, J. M. Bishop, Proc Natl Acad Sci USA 99, 5442 (2002).
54. K. R. Thomas, M. R. Capecchi, Cell 51, 503 (1987).
55. S. L. Mansour, K. R. Thomas, M. R. Capecchi, Nature 336, 348 (1988).
56. G. E. Truett et al., Biotechniques 29, 52 (2000).
57. G. J. Todaro, H. Green, J Cell Biol 17, 299 (1963).
58. H. Sasaki, C. Hui, M. Nakafuku, H. Kondoh, Development 124, 1313 (1997).
59. J. Taipale et al., Nature 406, 1005 (2000).
60. J. Svärd et al., Dev Cell 10, in press (2006).
61. M. L. Brinkmeier et al., Mol Endocrinol 12, 622 (1998).
62. P. Maye, S. Becker, E. Kasameyer, N. Byrd, L. Grabel, Mech Dev 94, 117 (2000).
63. F. Spreafico, J. J. Barski, C. Farina, M. Meyer, Mol Cell Neurosci 17, 1 (2001).
64. P. Chomczynski, N. Sacchi, Anal Biochem 162, 156 (1987).
65. D. Henrique et al., Nature 375, 787 (1995).
66. L. V. Goodrich, R. L. Johnson, L. Milenkovic, J. A. McMahon, M. P. Scott, Genes Dev 10, 301 (1996).
67. T. Grimm et al., FEBS Lett 505, 13 (2001).
68. D. Zechner et al., Dev Biol 258, 406 (2003).
69. J. Briscoe, A. Pierani, T. M. Jessell, J. Ericson, Cell 101, 435 (2000).
70. T. Yamada, S. L. Pfaff, T. Edlund, T. M. Jessell, Cell 73, 673 (1993).
71. J. Jensen, P. Serup, C. Karlsen, T. F. Nielsen, O. D. Madsen, J Biol Chem 271, 18749 (1996).
72. S. Thor, J. Ericson, T. Brannstrom, T. Edlund, Neuron 7, 881 (1991).
73. N. Schaeren-Wiemers, A. Gerfin-Moser, Histochemistry 100, 431 (1993).
74. E. Segev, N. Halachmi, A. Salzberg, N. Ben-Arie, Mech Dev 106, 197 (2001).
75. G. Regl et al., Oncogene 21, 5529 (2002).
76. L. V. Goodrich, L. Milenkovic, K. M. Higgins, M. P. Scott, Science 277, 1109 (1997).
77. J. Briscoe, J. Ericson, Curr Opin Neurobiol 11, 43 (2001).
78. D. Stamataki, F. Ulloa, S. V. Tsoni, A. Mynett, J. Briscoe, Genes Dev 19, 626 (2005).
79. M. Placzek, J. Briscoe, Nat Rev Neurosci 6, 230 (2005).
80. Y. Jeong, D. J. Epstein, Development 130, 3891 (2003).
81. C. B. Bai, W. Auerbach, J. S. Lee, D. Stephen, A. L. Joyner, Development 129, 4753 (2002).
82. J. K. Chen, J. Taipale, K. E. Young, T. Maiti, P. A. Beachy, Proc Natl Acad Sci USA 99, 14071 (2002).
83. M. E. Hutchin et al., Genes Dev 19, 214 (2005).
84. V. Grachtchouk et al., EMBO J 22, 2741 (2003).

1. A cell-based assay system for identifying an inhibitor and/or an antagonist of a Hedgehog signalling pathway, comprising cells, excluding human embryonic stem cells, which lack a functional Sufu protein.

2. A cell-based assay system according to claim 1, wherein said cells are Sufu−/− cells.

3. A cell-based assay system according to claim 1, wherein said cells are mammalian cells.

4. A cell-based assay system according to claim 2, wherein said cells are mouse cells.

5. A cell-based assay system according to claim 4, wherein said cells are mouse Sufu−/− embryonic fibroblasts.

6. A method for identifying an inhibitor and/or an antagonist of a Hedgehog signalling pathway, which method comprises using a cell-based system comprising cells, excluding human embryonic stem cells, which lack a functional Sufu protein.

7. A method according to claim 6, wherein said cell-based system comprises Sufu−/− cells.

8. A method according to claim 6, wherein said cell-based system comprises mammalian cells.

9. A method according to claim 8, wherein said cell-based system comprises mouse cells.

10. A method according to claim 9, wherein said cell-based system comprises mouse Sufu−/− embryonic fibroblasts.

11. A method according to claim 6, wherein said cell-based system comprises cells selected from the group consisting of prokaryotic cells, eukaryotic cells, insect cells, and yeast cells.

12. A method according to claim 6, which method comprises:
a) adding a substance to be tested to a cell-culture comprising cells lacking a functional Sufu protein, and;
b) detecting whether or not said substance has an inhibiting and/or antagonizing function on a Hedgehog signalling pathway in said cells.

13. A method according to claim 12, wherein cells lacking a functional Sufu protein in step a) are generated by deleting the Sufu gene from the genome in said cells.

14. A method according to claim 6, which method comprises the following steps:
a) adding a substance to be tested to Sufu^{-/-} cells transfected with a reporter gene system responsive to a Hedgehog signalling gene product;
b) lysing said cells, and;
c) measuring the levels of reporter gene expression and/or activity to detect whether or not said substance has an inhibiting and/or antagonizing function on a Hedgehog signalling pathway in said cells.

15. A method according to claim 14, wherein said Sufu^{-/-} cells in step a) are generated by deleting the Sufu gene from the genome in said cells.

16. A method according to claim 14, wherein said reporter gene system in step a) comprises a plasmid for normalization.

17. A method according to claim 14, wherein in step a) said reporter gene system is responsive to Gli-mediated transcription of a gene.

18. A method according to claim 14, wherein said reporter gene system comprises a reporter gene comprising Gli binding sites.

19. A method according to claim 14, wherein said reporter gene system comprises 8 Gli binding sites and a phRL-SV40 or a phRL-TK plasmid.

20. A method according to claim 14, wherein said detection in step c) is performed with a luciferase reporter system.

21. A method according to claim 6, which method comprises the following steps:
a) adding a substance to be tested to untransfected confluent Sufu^{-/-} cells;
b) preparing DNAse-treated RNA from said cells and reversibly transcribing said RNA into cDNA;
c) performing real-time PCR with said cDNA for the detection of Hedgehog target gene mRNA, and;
d) normalizing the level of a Hedgehog target gene mRNA against the level of a housekeeping gene mRNA using a relative and/or quantitative method to detect whether or not said substance has an inhibiting and/or antagonizing function on a Hedgehog signalling pathway in said cells.

22. A method according to claim 21, wherein said Hedgehog target gene mRNA in step c) is Gli1 mRNA.

23. A method according to claim 21, wherein said normalisation in step d) is performed against Gapdh mRNA levels using a standard curve of a Gli1 positive sample to detect whether or not said substance has an inhibiting and/or antagonizing function on a Hedgehog signalling pathway.

24. A method according to claim 6, for identifying an inhibitor and/or an antagonist of a Hedgehog signalling pathway downstream of Smo and Ptch.

25. A method for preparing an immortalized mouse embryo fibroblast Sufu^{-/-} cell culture, which method comprises the following steps:
a) intercrossing chimeric Sufu^{+/-} mice comprising a vector that targets a Sufu locus to generate Sufu^{-/-} mice embryos;
b) selecting Sufu^{-/-} embryos, and;
c) incubating cells obtained from said Sufu^{-/-} mice embryos in MEF medium, thereby preparing said immortalized mouse embryo fibroblast Sufu^{-/-} cell culture.

26. A method according to claim 25, wherein said vector in step a) is pSufuΔexon1neo.

27. A method according to claim 6, wherein said cells that lack a functional Sufu protein are immortalized mouse embryo fibroblast Sufu^{-/-} cells.

28. A cell culture prepared according to claim 25.

29. A cell culture comprising Sufu^{-/-} cells, provided that said cells are not human embryonic stem cells.

30. A method according to claim 12, wherein said cells that lack a functional Sufu protein are immortalized mouse embryo fibroblast Sufu^{-/-} cells.

31. A method according to claim 12, wherein step b) comprises detecting whether or not said substance has an inhibiting and/or antagonizing function on a Hedgehog signalling pathway downstream of Smo and Ptch.

32. A method according to claim 30, wherein said step b) comprises detecting whether or not said substance induces cell death by necrosis or apoptosis in said cells.

35. A method according to claim 6, said method comprising using said cells to test whether or not a substance has an inhibiting and/or antagonizing function on a Hedgehog signalling pathway downstream of Smo and Ptch.

36. A transgenic mouse lacking a functional Sufu protein.


SEQUENCE LISTING (30 sequences)

SEQ ID NO: 1 - DNA, 20 bp, Artificial Sequence
  primer
ccctttttgtcaatagttcc

SEQ ID NO: 2 - DNA, 20 bp, Artificial Sequence
  primer
tgacaatagactccgcctcc

SEQ ID NO: 3 - DNA, 21 bp, Artificial Sequence
  primer
gccttctatcgccttcttgac

SEQ ID NO: 4 - DNA, 20 bp, Artificial Sequence
  primer
ccctttttgtcaatagttcc

SEQ ID NO: 5 - DNA, 20 bp, Artificial Sequence
  primer
tgacaatagactccgcctcc

SEQ ID NO: 6 - DNA, 20 bp, Artificial Sequence
  primer
ccctttttgtcaatagttcc

SEQ ID NO: 7 - DNA, 21 bp, Artificial Sequence
  primer
gccttctatcgccttcttgac

SEQ ID NO: 8 - DNA, 20 bp, Artificial Sequence
  primer
gcaaaaccacactgctcgac

SEQ ID NO: 9 - DNA, 29 bp, Artificial Sequence
  primer
ttctcccccaacttctgctgccaatctcc

SEQ ID NO: 10 - DNA, 23 bp, Artificial Sequence
  primer
agtatggctcattggttcttggg

SEQ ID NO: 11 - DNA, 23 bp, Artificial Sequence
  primer
ctccccttgcctggtctgtgtgt

SEQ ID NO: 12 - DNA, 30 bp, Artificial Sequence
  primer
ttcactggccgtcgttttacaacgtcgtga

SEQ ID NO: 13 - DNA, 30 bp, Artificial Sequence
  primer
atgtgagcgagtaacaacccgtcggattct

SEQ ID NO: 14 - DNA, 17 bp, Artificial Sequence
  primer
gaccgggactatctgca

SEQ ID NO: 15 - DNA, 19 bp, Artificial Sequence
  primer
ctcctatcttctgacgggt

SEQ ID NO: 16 - DNA, 20 bp, Artificial Sequence
  primer
tccaaggtctactcttctcc

SEQ ID NO: 17 - DNA, 20 bp, Artificial Sequence
  primer
gctcctcgagcagctgctga

SEQ ID NO: 18 - DNA, 24 bp, Artificial Sequence
  primer
tgggatccagtgccagaacccgct

SEQ ID NO: 19 - DNA, 24 bp, Artificial Sequence
  primer
acggtaccgatagttcttgtagcc

SEQ ID NO: 20 - DNA, 21 bp, Artificial Sequence
  primer
ctccaggttaccgctatcgtc

SEQ ID NO: 21 - DNA, 21 bp, Artificial Sequence
  primer
cacttggtccgtctgttcctg

SEQ ID NO: 22 - DNA, 20 bp, Artificial Sequence
  primer
ttcgtgtgccattggggagg

SEQ ID NO: 23 - DNA, 20 bp, Artificial Sequence
  primer
cttgggctccactgtggaga

SEQ ID NO: 24 - DNA, 20 bp, Artificial Sequence
  primer
ttcgtgtgccgctggcaggc

SEQ ID NO: 25 - DNA, 20 bp, Artificial Sequence
  primer
ttgagcagtggagcacggac

SEQ ID NO: 26 - DNA, 20 bp, Artificial Sequence
  primer
tacaggccagactttgttgg

SEQ ID NO: 27 - DNA, 20 bp, Artificial Sequence
  primer
aacttgcgctcatcttaggc

SEQ ID NO: 28 - DNA, 21 bp, Artificial Sequence
  primer
gaccgggctggcagttccttc

SEQ ID NO: 29 - DNA, 23 bp, Artificial Sequence
  primer
tgcgttccatgtcgttccagtcc

SEQ ID NO: 30 - DNA, 380 bp, Artificial Sequence
  probe sequence
gaattcaatcttcaactacttctctgctggatttatggggaaatttaatgtgatccttaggagcctcaaatcgtgagatgtcccagctcttcctttgagcccagtgcctcccggcttactctttagaatgtaggtgctcatggggctcagcctggctctcgctgcagtgctctgtgcagagttgattgagccaatgtttggccttcgtcacttgcagcgactaagttttgatctgtggcgtataggccacgatctgtgcagactggcagctttatgtatatccagacgaaacaatgatagaaggcacagtacagttttttagaaactagctgtctaagagtcaggtgcagagtggaacctgaactttcaggaatgatatc